(19) United States
(12) Patent Application Publication
(10) Pub. No.: US 2024/0000322 A1
(43) Pub. Date: Jan. 4, 2024

(54) ADVANCED MECHANO-ACOUSTIC SENSING AND APPLICATIONS OF SAME

(71) Applicant: NORTHWESTERN UNIVERSITY, Evanston, IL (US)

(72) Inventors: Shuai Xu, Bala Cynwyd, PA (US); Hyoyoung Jeong, Evanston, IL (US); Jong Yoon Lee, Evanston, IL (US); Kun Hyuck Lee, Evanston, IL (US); John A. Rogers, Wilmette, IL (US)

(21) Appl. No.: 18/034,893

(22) PCT Filed: Nov. 2, 2021
(86) PCT No.: PCT/US2021/057686

§ 371 Date: May 2, 2023

Related U.S. Application Data
(63) Continuation-in-part of application No. 16/970,023, filed on Aug. 14, 2020.
(60) Provisional application No. 63/108,514, filed on Nov. 2, 2020.
(60) Provisional application No. 62/753,203, filed on Oct. 31, 2018.
(60) Provisional application No. 62/631,692, filed on Feb. 17, 2018.
(60) Provisional application No. 62/710,324, filed on Feb. 16, 2018.

(51) Int. Cl. A61B 5/0205; A61B 5/113; A61B 5/00

(52) U.S. Cl. CPC A61B 5/02055; A61B 5/1135; A61B 5/7214; A61B 2562/0219; A61B 5/6832; A61B 5/4803; A61B 5/4205

(57) ABSTRACT

This invention discloses an electronic device for measuring physiological parameters of a living subject including at least a first inertial measurement unit (IMU) and a second IMU, the first IMU and the second IMU are time-synchronized to and spatially and mechanically separated from each other; and a microcontroller unit (MCU) electronically coupled to the first IMU and the second IMU for processing of data streams from the first IMU and the second IMU.

### CROSS-REFERENCE TO RELATED PATENT APPLICATIONS

This application claims priority to and the benefit of U.S. Provisional Patent Application Ser. No. 63/108,514, filed Nov. 2, 2020.

This application is also a continuation-in-part application of U.S. patent application Ser. No. 16/970,023, filed Aug. 14, 2020, which is a national stage entry of PCT Patent Application Serial No. PCT/US2019/018318, filed Feb. 15, 2019, which itself claims priority to and the benefit of U.S. Provisional Patent Application Serial Nos. 62/710,324, filed Feb. 16, 2018, 62/631,692, filed Feb. 17, 2018, and 62/753,203, filed Oct. 31, 2018.

Each of the above-identified applications is incorporated herein by reference in its entirety.

### STATEMENT AS TO RIGHTS UNDER FEDERALLY-SPONSORED RESEARCH

This invention was made with government support under 75A50119C00043 awarded by the Office of the Assistant Secretary for Preparedness and Response, and AG062023 and AG060812 awarded by the National Institutes of Health. The government has certain rights in the invention.

### FIELD OF THE INVENTION

The present invention relates generally to biosensors, and more particularly to advanced mechano-acoustic sensing systems and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

The emergence of wearable technologies capable of multimodal, clinical-grade monitoring of physiological health increases the demand for sensors, systems, and data analytics approaches that enable reliable, continuous operation during natural daily activities. By comparison to traditional devices that loosely couple to the wrist, skin-mounted technologies offer vastly superior measurement capabilities due to their persistent, intimate interfaces to the body. This mode of operation can support a range of clinically standard diagnostic assessments, such as those based on electrocardiography, photoplethysmography, arterial tonometry, and others. A recent set of important capabilities follows from wide-bandwidth measurements of subtle motions and vibrations of the surface of the skin (i.e., mechano-acoustic (MA) responses) that arise from activities of internal organs and accelerations due to global movements of the body. Skin-interfaced devices for such purposes use precision, high-bandwidth accelerometers based on microelectromechanical system technologies in layouts that optimize sensitivity to motions of the surface of the skin across a broad range of frequencies, from nearly zero to several thousand hertz. The resulting data reflect not only bulk motions of the body, as with conventional wearable devices, but also features from a broad range of body sounds, as with digital stethoscopes, but impervious to ambient sounds. Additional information appears in a range of frequencies between these limits. For example, when mounted on the neck or the chest, the recordings enable detailed assessments of cardiac activity from motions of the heart and from pulsatile flow of blood through near-surface arteries, of respiratory cycles from chest wall movements, of respiratory sounds from airflow through the lungs and trachea, of swallowing behaviors from laryngeal motions and actions of the esophagus, of vocalization patterns from vocal fold activation, and of movements and changes in orientation of the core body. Distinct features in the temporal and spectral characteristics of these processes yield insights into physical activity and health status via a rich range of conventional (e.g., heart rate (HR)) and unconventional (e.g., coughing frequency) metrics, in a seamless manner, without privacy concerns that would follow from use of microphones or other recording devices.

Through these mechanisms, a single device in a sealed, waterproof package that requires only mechanical coupling to the skin can produce a powerful breadth of health-related information. An important caveat is that the diverse range of MA signals contribute to a single stream of time series data in a temporally overlapping fashion. Advanced data filtering and analytics approaches can separate and quantify different characteristic events on the basis of unique temporal and spectral features, but they fail to operate reliably in many scenarios of practical interest. Particular challenges arise when different activities with similar spectral content occur simultaneously. These circumstances render digital signal processing approaches ineffective. For example, respiration rate cannot be determined accurately while running. Related types of motion artifacts are fundamental limitations to both consumer wearables mounted on the wrist and clinical-grade wired monitoring systems.

Therefore, a heretofore unaddressed need exists in the art to address the aforementioned deficiencies and inadequacies.

### SUMMARY OF THE INVENTION

This invention in certain aspects discloses a novel approach that overcomes the aforementioned limitations through advanced concepts in system designs and optimized choices in anatomical mounting locations, at the hardware level without the need for complex and often ineffective digital signal processing strategies. The approach in some embodiments exploits a pair of time-synchronized, high-bandwidth accelerometers (inertial measurement units (IMUs)) at opposite ends of a skin-interfaced device that locates one of the IMUs at the suprasternal notch (SN) and the other at the sternal manubrium (SM). Differences in movements of the skin associated with cardiac and respiratory activity between these regions lead to differences in signals captured by these IMUs. By contrast, overall movements of the neck and the core of the body produce nearly identical responses. As a consequence, simple differential measurements cleanly eliminate common mode features, thereby separating signals associated with cardiopulmonary and related processes from those due to body movements. An additional benefit of this architecture is that temperature sensors integrated in these IMUs can be used in a similar differential manner to yield estimates of core body temperature, largely independent of the ambient. Here, careful choices in thermal aspects of the device layout, rather than intrinsic anatomical gradients, produce the necessary differential responses.

In one aspect, the invention relates to an electronic device for measuring physiological parameters of a living subject comprising at least a first IMU and a second IMU, the first IMU and the second IMU are time-synchronized to and spatially and mechanically separated from each other; and a microcontroller unit (MCU) electronically coupled to the first IMU and the second IMU for processing of data streams from the first IMU and the second IMU.

In one embodiment, the first IMU is configured to measure data including a first signal related to a physiological signal of the living subject and a second signal, and the second IMU is configured to measure data including at least the second signal. The first signal measured by the first IMU has a signal strength greater than that the second signal measured by the first IMU.

In one embodiment, the data measured by the first IMU and the second IMU are processed such that subtraction of the second signal measured by the second sensor from the second signal measured by the first sensor results in a stronger first signal that is a signal of interest.

In one embodiment, the second signal is related to at least one of ambient, motion and vibration.

In one embodiment, the data measured by the second IMU includes the first signal and the second signal.

In one embodiment, a signal-to-noise ratio (SNR) of a signal measured by the first IMU and the second IMU together is lower than a first SNR of a signal measured by the first IMU individually, or a second SNR of a signal measured by the second IMU individually.

In one embodiment, both of the first IMU and the second IMU are operably in mechanical communication with the skin of the living subject.

In one embodiment, one of the first IMU and the second IMU is operably in directly mechanical communication with the skin of the living subject for sensing physiological signals of the body, while the other of the first IMU and the second IMU is operably in indirectly mechanical communication with the skin of the living subject.

In one embodiment, the first IMU and the second IMU are operably in directly mechanical communication with the skin of the living subject.

In one embodiment, one of the first IMU and the second IMU is separated from the rest of rigid components of the electronic device.

In one embodiment, the electronic device also comprises at least first and second thermal sensing units, wherein one of the first and second thermal sensing units is thermally isolated from an ambient environment and configured to measure a body temperature of the living subject, and the other of the first and second thermal sensing units is configured to measure the ambient temperature.

In one embodiment, each of the first and second thermal sensing units is embedded in a respective one of the first and second IMUs.

In one embodiment, the electronic device is configured to measure a range of physiological information from activity of a cardiopulmonary system and movements of a core body to a diverse collection of processes across thoracic cavity, esophagus, pharynx, and oral cavity related to respiration, speech, swallowing, wheezing, coughing, and sneezing.

In one embodiment, the electronic device is configured to separate signals associated with the cardiopulmonary system and related processes from those due to body movements.

In one embodiment, the electronic device is configured to spatiotemporally map movements of the skin at this region of the anatomy onto which the electronic device is attached during cardiac and respiratory activities.

In one embodiment, the electronic device is configured to continuously measure temperature, heart rate (HR), respiratory rate (RR), activity level, and body orientation, across a range of vigorous activities and conditions.

In one embodiment, the electronic device is configured to monitor key symptoms of a patient with COVID-19 infection to track progress of recovery and response to therapies in hospital and/or home.

In one embodiment, the electronic device is configured to measure any of respiratory or motion related digital biomarkers associated with coughing, swallowing, and/or specific motion related activities.

In one embodiment, the electronic device is configured to assess coughing when the living subject is moving or immobile, and/or to measure muscle motion, when the living subject is moving.

In one embodiment, the electronic device further comprises a bidirectional wireless communication system electronically coupled to the electronic device and configured to send an output signal from the electronic device to an external device.

In one embodiment, the external device is a mobile device, a computer, or a cloud service.

In one embodiment, the bidirectional wireless communication system is further configured to deliver commands from the external device to the electronic device.

In one embodiment, the bidirectional wireless communication system comprises a controller that utilizes at least one of near field communication (NFC), Wi-Fi/Internet, Bluetooth, Bluetooth low energy (BLE), and cellular communication protocols for wireless communication.

In one embodiment, the electronic device further comprises a customized app with a user interface deployed in the external device to allow a user to configure and operate the electronic device for data collection, data transfer, data storage and analysis, wireless charging, and monitoring of user's conditions.

In one embodiment, the customized app is configured to allow time-synchronized operation of a plurality of the electronic devices simultaneously.

In one embodiment, the electronic device further comprises a power module coupled to the first IMU, the second IMU and the MCU for providing power thereto.

In one embodiment, the power module comprises at least one battery for providing the power. In one embodiment, the battery is a rechargeable battery.

In one embodiment, the power module further comprises a wireless charging module for wirelessly charging the rechargeable battery.

In one embodiment, the power module further comprises a failure prevention element including a short-circuit protection component or a circuit to avoid battery malfunction.

In one embodiment, the second IMU is placed in a manner that it bends and folds over the battery.

In one embodiment, the electronic device further comprises a flexible printed circuit board (fPCB) having flexible and stretchable interconnects electrically connecting to electronic components including the first IMU, the second IMU and the MCU and the power module.

In one embodiment, the electronic device further comprises an elastomeric encapsulation layer at least partially surrounding the electronic components and the flexible and stretchable interconnects to form a tissue-facing surface attached to the living subject and an environment-facing surface, wherein the tissue-facing surface is configured to conform to a skin surface of the living subject.

In one embodiment, the encapsulation layer is formed of a flame retardant material.

In one embodiment, the elastomeric encapsulation layer is a waterproof and biocompatible silicone enclosure.

In one embodiment, the electronic device further comprises a biocompatible hydrogel adhesive for attaching the electronic device on the respective region of the living subject, wherein the biocompatible hydrogel adhesive is adapted such that signals from the living subject are operably conductible to the first IMU and the second IMU.

In one embodiment, the electronic device is flexible and conformable to the skin with a specific geometrical polarity for mounting in an anatomical location of interest of the living subject.

In one embodiment, the electronic device is a wearable, twistable stretchable, and/or bendable.

In another aspect, the invention relates to an electronic device for measuring physiological parameters of a living subject, comprising a sensor network comprising a plurality of sensor units operably deployed on a skin of the living subject, the plurality of sensor units being time-synchronized to and spatially and mechanically separated from each other; and an MCU electronically coupled to the plurality of sensor units for processing of data streams from the plurality of sensor units.

In one embodiment, the plurality of sensor units are configured to measure a same physiological parameter, or different physiological parameters.

In one embodiment, each of the plurality of sensor units comprises at least a first sensor and the second sensor time-synchronized to and spatially and mechanically separated from each other.

In one embodiment, for each sensor unit, the first sensor is configured to measure data including a first signal related to a physiological signal of the living subject and a second signal, and the second sensor is configured to measure data including at least the second signal. The first signal measured by the first sensor has a signal strength greater than that the second signal measured by the first sensor.

In one embodiment, the data measured by the first sensor and the second sensor of said sensor unit are processed such that subtraction of the second signal measured by the second sensor from the second signal measured by the first sensor results in a stronger first signal that is a signal of interest

In one embodiment, the second signal is related to at least one of ambient, motion and vibration.

In one embodiment, each of the first sensor and the second sensor comprises the IMU, a thermal sensor, a pressure sensor, and/or optical sensor.

In one embodiment, each of the first sensor and the second sensor comprises the IMU.

In one embodiment, the electronic device further comprises a plurality of thermal sensing units.

In one embodiment, each thermal sensing units is embedded in a respective IMU.

In one embodiment, the MCU operably receives inputs from synchronized outputs of a plurality of thermal sensor units with at least one thermal sensing unit for the ambient environment and at least one thermal sensing unit in direct thermal communication from the body isolated thermally from the ambient environment with in-sensor thermally isolating materials.

In one embodiment, the electronic device is configured to automatically switch operation modes, the operation modes include at least a first mode when the living subject is at rest, and a second modes when the living subject is in a high motion.

In one embodiment, the electronic device is configured to measure a range of physiological information from activity of a cardiopulmonary system and movements of a core body to a diverse collection of processes across thoracic cavity, esophagus, pharynx, and oral cavity related to respiration, speech, swallowing, wheezing, coughing, and sneezing.

In one embodiment, the electronic device is configured to separate signals associated with the cardiopulmonary system and related processes from those due to body movements.

In one embodiment, the electronic device is configured to spatiotemporally map movements of the skin at this region of the anatomy onto which the electronic device is attached during cardiac and respiratory activities.

In one embodiment, the electronic device is configured to continuously measure temperature, heart rate (HR), respiratory rate (RR), activity level, and body orientation, across a range of vigorous activities and conditions.

In one embodiment, the electronic device is configured to monitor key symptoms of a patient with COVID-19 infection to track progress of recovery and response to therapies in hospital and/or home.

In one embodiment, the electronic device is configured to measure any of respiratory or motion related digital biomarkers associated with coughing, swallowing, and/or specific motion related activities.

In one embodiment, the electronic device is configured to assess coughing when the living subject is moving or immobile, and/or to measure muscle motion, when the living subject is moving.

In one embodiment, the electronic device further comprises a bidirectional wireless communication system electronically coupled to the electronic device and configured to send an output signal from the electronic device to an external device.

In one embodiment, the external device is a mobile device, a computer, or a cloud service.

In one embodiment, the bidirectional wireless communication system is further configured to deliver commands from the external device to the electronic device.

In one embodiment, the bidirectional wireless communication system comprises a controller that utilizes at least one of NFC, Wi-Fi/Internet, Bluetooth, BLE, and cellular communication protocols for wireless communication.

In one embodiment, the electronic device further comprises a customized app with a user interface deployed in the external device to allow a user to configure and operate the electronic device for data collection, data transfer, data storage and analysis, wireless charging, and monitoring of user's conditions.

In one embodiment, the customized app is configured to allow time-synchronized operation of a plurality of the sensor network simultaneously.

In one embodiment, the electronic device further comprises a power module coupled to the sensor network for providing power thereto.

In one embodiment, the power module comprises at least one battery for providing the power. In one embodiment, the battery is a rechargeable battery.

In one embodiment, the power module further comprises a wireless charging module for wirelessly charging the rechargeable battery.

In one embodiment, the power module further comprises a failure prevention element including a short-circuit protection component or a circuit to avoid battery malfunction.

In yet another aspect, the invention relates to an electronic device for measuring physiological parameters of a living subject, comprising a first sensor adapted for detecting a first group of data related to the living subject and a second group of data that is different from the first group of data; and a second sensor for detecting a third group of data that is substantially similar to the second group of data. In operation, the first sensor and the second sensor are time-synchronized to allow the third group of data from the second sensor to be used to substantially cancel out the second group of data from the first sensor.

In one embodiment, the first sensor and the second sensor are spatially and mechanically separated from each other.

In one embodiment, the separation of the first sensor and the second sensor is greater than zero and less than a predetermined distance.

In one embodiment, each of the first sensor and the second sensor comprises an IMU, a thermal sensor, a pressure sensor, or optical sensor.

In one embodiment, the first group of data is physiological signals of the living subject, and the second group of data is ambient signals at the first sensor.

In one embodiment, the third group of data is ambient signals at the second sensor.

In one embodiment, both of the first sensor and second sensor are operably in mechanical communication with the skin of the living subject.

In one embodiment, the first sensor is operably in direct mechanical communication with the skin of the living subject for sensing physiological signals from the body, and the second sensor is operably in indirectly mechanical communication with the skin of the living subject.

In one embodiment, the first sensor and the second sensor are operably in directly mechanical communication with the skin of the living subject for sensing physiological signals from the body to assess pulse transit time.

In one embodiment, the electronic device is flexible and conformable to the skin with a specific geometrical polarity for mounting in an anatomical location of interest of the living subject.

In a further aspect, the invention relates to an electronic device for measuring physiological parameters of a living subject, comprising a first sensor adapted for detecting a first group of data related to the living subject and a second group of data that is different from the first group of data; and a second sensor for detecting a third group of data that is substantially similar to the second group of data, wherein in operation, the first sensor is positioned such that there is a first distance d1 between a center of the first sensor and an area of the living subject where physiological signals of the living subject are measurable; the second sensor is positioned such that there is a second distance d2 between a center of the second sensor and the center of the first sensor, wherein the second distance d2 is greater than zero and less than a predetermined distance.

In one embodiment, the second sensor is positioned over the first sensor.

In one embodiment, the second sensor is positioned away from the first sensor.

In one embodiment, each of the first sensor and the second sensor comprises an IMU, a thermal sensor, or a pressure sensor.

In one embodiment, the first group of data is physiological signals of the living subject, and the second group of data is signals related to ambient, motion and/or vibration at the first sensor.

In one embodiment, the third group of data is signals related to ambient, motion and/or vibration at the second sensor.

In one embodiment, both of the first sensor and second sensor are operably in mechanical communication with the skin of the living subject.

In one embodiment, the first sensor is operably in directly mechanical communication with the skin of the living subject for sensing physiological signals from the body, and the second sensor is operably in indirectly mechanical communication with the skin of the living subject.

In one embodiment, both of the first sensor and the second sensor are operably in directly mechanical communication with the skin of the living subject for sensing physiological signals from the body to assess pulse transit time.

In one embodiment, the electronic device is flexible and conformable to the skin with a specific geometrical polarity for mounting in an anatomical location of interest of the living subject.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIGS. 1A-1F show images, schematic illustrations, functional flow charts, and mechanical modeling results for a wireless, skin-interfaced device designed for dual MA measurements at the SN and the SM, according to embodiments of the invention. FIG. 1A: Image of the device mounted on the base of the neck, positioned with one end at the SN and the other at the SM. FIG. 1B: Exploded-view schematic illustration of the active components, interconnect schemes, and enclosure architectures. FIG. 1C: Image of a device next to a U.S. quarter (diameter, 24.26 mm). FIG. 1D: Images of the device during various mechanical deformations: a twisting angle of 90° (left), 45% uniaxial stretching (middle), and a bending angle of 180° (right). FIG. 1E: Finite element modeling of the mechanics for the deformations in FIG. 1D. The contour plots show the maximum principle strains in the metal layer of the serpentine interconnects for twisting (left), stretching (middle), and bending (right). FIG. 1F: Block diagram of the system operation. A tablet provides an interface for operating the device, wirelessly downloading the data from the device, and transmitting these data to a cloud server through a cellular network. Processing on the cloud platform yields vital signals (HR, respiration, and body temperature) and other metrics of interest (cough count and physical activity).

FIGS. 2A-2G show a dual-sensing platform for differential temperature and MA sensing, according to embodiments of the invention. FIG. 2A: Exploded-view and FIG. 2B: cross-sectional schematic illustrations of the device. FIG. 2C: Side view of a completed device next to a U.S. quarter. FIG. 2D: Finite element results for the temperature distribution in the skin and outside the device for skin and ambient temperatures of 37° and 22° C., respectively, with a convection coefficient of 10 W m′ K″. Cross-sectional profile of temperature along the A-B axis (inset). FIG. 2E: Temperature profile along the A-B cross section for different ambient temperatures and convection coefficients. FIG. 2F: Differential temperature measured using the temperature sensors in IMU1 and IMU2. (D) to (F) correspond to the case of a core body temperature of 37° C. FIG. 2G: Representative results determined as the subject moves through rooms at various ambient temperatures. Dual temperatures (first row), differential temperature (second row), and the calibrated and measured core body temperatures (third row).

FIGS. 3A-3H show distributions of displacements across the neck and surrounding regions determined by 3D-PTV during natural respiratory and cardiac activities, with a focus on the SN and the SM, according to embodiments of the invention. FIG. 3A: 3D vector and contour fields of displacements, superimposed on the neck image. FIG. 3B: 3D view of FIG. 3A. The color denotes the velocity along the z axis, w, during a cardiac cycle. FIG. 3C: Displacement along the z axis, ΔZ, as a function of time at the SN and SM during a breath hold, highlighting cardiac activity. FIG. 3D: Differential displacement between the SN and SM determined from the data in FIG. 3C. FIG. 3E: Color contour of ΔZ at the peak of a cardiac cycle highlighted by the blue arrow in FIG. 3D. FIG. 3F: AZ as a function of time at the SN and SM during breathing and slight body motions. FIG. 3G: Differential displacement between the SM and SN determined from the data in FIG. 3F. FIG. 3H: Color contour of ΔZ at the peak of inhalation, highlighted by the blue arrow in FIG. 3G.

FIGS. 4A-4D show representative data collected during various ambulatory motions and measurements of controlled RR and normal HR, according to embodiments of the invention. FIG. 4A: The subject sat quietly for 7 min, walked for 14 min with resting intervals, ran for 8 min with resting intervals, and jumped for 7 min with resting intervals under controlled RRs (6 to 35 RPM). FIG. 4B: Magnified views of walking and running signals from (A), highlighting baseline fluctuations associated with respiration. The far-right box in green outline is a further magnified view from the data in the middle frame, highlighting cardiac activities 51 and S2. FIG. 4C: Single-accelerometer data (black dot) yield reliable values of RR while the subject sits still. During ambulatory motions, the single-accelerometer data yield unreliable values of RR. The differential signals (blue dots) yield accurate respiration rates, consistent with ground truth (green triangles). The red arrow indicates the time frame of FIG. 4B. FIG. 4D: Single-accelerometer data provide the HR reliably while the subject sits still. During ambulatory motions, the single-accelerometer data (black dot) yield unreliable values compared to those from differential signals (blue dot) and from ground truth (green triangle). Signals associated with tapping between transitions cause aberrant values. The red arrow indicates the time frame of FIG. 4B.

FIGS. 5A-5H show tracking of cardiopulmonary activity during intense physical activities, according to embodiments of the invention. FIG. 5A: Image of the dual-sensing device at the SN/SM along with reference devices for SpO₂ and electrocardiogram recording and thermocouples for oral and ambient temperature measurements while cycling. FIG. 5B: Comparisons of RR and HR determined by the dual-sensing (blue square) and single-sensing (red circle) and reference devices (green triangle, for HR only) while cycling for 24 min. FIG. 5C: Image of the dual-sensing device on the SN/SM while playing basketball. FIG. 5D: Comparisons of RR and HR determined from the dual- and single-sensing data while playing basketball for 11 min. FIG. 5E: Image of the dual-sensing device on the SN/SM while swimming. FIG. 5F: Comparisons of RR and HR determined with the dual- and single-sensing data while swimming for 5 min. FIG. 5G: Representative z-axis acceleration data acquired from the dual-sensing device during swimming. Accelerations measured from IMU1 (red), IMU2 (black), calculated differential signal (blue), and baseline of the differential signal (light blue). FIG. 5H: Magnified data associated with the differential signal (blue) and its baseline (light blue) from the area highlighted by the green box FIG. 5G.

FIGS. 6A-6D show data collected from a COVID-19 patient in the form of cough count, RR, HR, activity level, and estimated core body temperature, according to embodiments of the invention. FIG. 6A: Variation of cough frequency from the patient while recovering over a period of 8 days. The first set was measured from 1 to 7 p.m. on the first day. The second set was measured from 8 a.m. to 8 p.m. on the second day. The third set was measured from 1 to 9 p.m. on the fourth day. The fourth set was measured from 9 a.m. to 8 p.m. on the seventh day, and the fifth set was measured from 8 a.m. to 8 p.m. on the eighth day. The purple line shows the cumulative number of coughs. FIG. 6B: Variation of respiration rate and results from Savitzky-Golay smoothing (orange line). FIG. 6C: Variation of HR and results from Savitzky-Golay smoothing (red line). FIG. 6D: Activity level (green bar) and estimated core body temperature (red) during day (yellow shaded region) and night (blue shaded region). a.u., arbitrary units.

FIG. 7 shows devices under CDC guided cleaning and disinfecting process with 70% alcohol solution.

FIG. 8 shows a layout of the flexible PCB for the dual-sensing device, according to embodiments of the invention. Red dashed lines show the folding planes and yellow dashed line shows the actual device size after folding and encapsulation.

FIG. 9 shows time synchronized operation of 12 devices for whole body acceleration measurements, according to embodiments of the invention.

FIG. 10 shows a dual-sensing system state diagram, according to embodiments of the invention.

FIG. 11 shows temperature profile of IMU1 and IMU2 along A-B cross-section under different core body temperatures (T_{core}=37, 38, 39, 40° C.).

FIG. 12 shows temperature difference between IMU1 and IMU2 under different ambient temperatures and convection coefficients (T_{core}=37, 38, 39, 40° C.).

FIG. 13 shows temperature color mapping of IMU1 and IMU2 along A-B cross-section under different ambient temperatures (T_{amb}: from 18° C. to 24° C.) and convection coefficients (5 W/m²K to 30 W/m²K).

FIG. 14 shows a 1-D heat transfer model, according to embodiments of the invention. (A) Illustration of the heat transfer model. (B) Thickness and thermal conductivity of each material layer.

FIG. 15 shows representative dual-sensing data collected from a subject during movement through rooms at various ambient temperatures. Temperature discrepancy (line) and percent difference (bar) between the reference temperature measurement data (oral thermocouple) and the estimated core body temperature (dual temperature sensing).

FIG. 16 shows Bland-Altman plots for temperature difference between oral thermocouple and IMU1 (A) (n=299 data points from 1 subject) and calibrated temperature from differential temperature of IMU1 and IMU2 (B) (n=359 data points from 1 subject).

FIG. 17 shows temperature results from the 1-D analytical model and 3-D FEA model. (A) IMU1 and IMU2 temperatures from the 1-D analytical model and the 3-D FEA model. (B) Analytical results determined with the temperature of IMU1, IMU2, differential, and a heat convection coefficient (h≈10 W/(m² k)) for changing ambient temperatures (T_{amb}) when the core temperature (T_{core}) remains at 36.3° C.

FIG. 18 shows 3D-PTV measurements. (A) Photograph and (B) illustration of the experimental setup. (C) Velocity along the z-axis, w vs time at the SN (IMU1 location) and SM (IMU2 location). (D) Color contours of (C), w at t=0.785 s (local maximum velocity) from the green dot in (C). (E) Displacements along the y-axis ΔY vs time at the SN and SM during breathing. (F) Differential displacement of (E) along the y-axis ΔY between the SM and SN, IMU2-IMU1. Photo credit: Jin-Tae Kim, Northwestern University.

FIG. 19 shows dual-sensing data collected during 3D-PTV measurements. (A) Acceleration along the z-axis vs time at the SN and SM during breathing. (B) Calculated velocity along the z-axis of (A). (C) Calculated displacement along the z-axis of (A). (D) Differential displacement: SM-SN of (C).

FIG. 20 shows simplified 1-D Analytical model of differential accelerometry. (A) Schematic illustration of the simplified 1-D Analytical Model for cardiac activity. A displacement is applied to the rigid platform causing sensors IMU1 and IMU2 (with mass m) to accelerate in the z-axis. IMU1 is tied to the platform and IMU2 is connected to the platform by a spring of stiffness k and damper with damping ratio (B) Analytical result of z-axis displacement from IMU1 and IMU2 during a cardiac cycle without respiratory activity. (C) Differential displacement between IMU1 and IMU2 determined from the data in (B). (D) Differential displacement between IMU1 and IMU2 determined from the data during respiratory activities.

FIG. 21 shows data collected from different body orientations with packaged and unpackaged devices. (A)-(D) Measured signal from an unpackaged dual-sensing device. (A) The subject sat quietly for 45 seconds, leaned back for 70 seconds, leaned forward for 35 seconds under normal and held breath conditions. (B) Magnified views of heart and respiratory activities from (A). (C) Flipped dual-sensing orientation. IMU1 was placed one the SM and IMU2 was placed on the SN. The subject sat quietly for 20 seconds, leaned back for 25 seconds, leaned forward for 30 seconds under normal and held breath conditions. (D) Magnified views of heart and respiratory activities from (C). (E)-(H) Measured signal from a packaged dual-sensing device. (E) The subject sat quietly for 20 seconds, leaned back for 45 seconds at two different angles, leaned forward for seconds under normal and held breath conditions. (F) Magnified views of heart and respiratory activities from (E). (G) Flipped dual-sensing orientation. IMU1 was placed one the SM and IMU2 was placed on the SN. The subject sat quietly for 20 seconds, leaned back for 45 seconds at two different angles, and leaned forward for 20 seconds under normal and held breath conditions. (H) Magnified views of heart and respiratory activities from (G).

FIG. 22 shows algorithm for determining the respiratory rate from the differential signal. (A) Block diagram of signal processing flow in the frequency domain. (B) Differential data derived from IMU1 and IMU2. Walking related impulse signals in IMU1 (red) and IMU2 (black) are largely absent from the differential data (blue). (C) Differential data in (B). (D) Fourier transform of (C). When the respiratory behavior is stable and constant, only one high-energy frequency appears in the range of 6-60 rpm. (E) Differential data for various respiratory rates. (F) Fourier transform of (E). Five high-energy frequencies appear in these data, three of which have more than 50% of the maximum energy. The respiratory rate corresponds to their weighted average according to the algorithm in (A).

FIG. 23 shows algorithm for determining the heart rate from the differential signal. (A) Block diagram of signal processing flow in frequency domain. (B) Differential data derived from IMU1 and IMU2. (C) Bandpass filtered signal of (B), with cutoff frequency in 45-170 bpm. (D) Detected envelope using an exponential function. (E) Fourier transform of the envelope data in (D).

FIG. 24 shows Bland-Altman plots for differences between respiratory rate (RR) from single sensing with IMU1 (A), IMU2 (B), and from the differential signal from IMU1 and IMU2 (C) (n=36 data points from 1 subject).

FIG. 25 shows Bland-Altman plots for differences between heart rate (HR) from single sensing with IMU1 (A), IMU2(B), and from the differential signal from IMU1 and IMU2 (C) (n=39 data points from 1 subject).

FIG. 26 shows measurement setup for the stationary bike riding. (A) subject on the bike wearing a sensor on the SN/SM. (B) Magnified image of the region highlighted by the orange box in (A).

FIG. 27 shows measured data from the stationary bike riding. (A) Z-axis acceleration while riding a stationary bike for 24 minutes. (B) Dual and reference temperature values while riding a stationary bike.

FIG. 28 shows measured data during push-ups with a controlled respiratory rate. (A) Z-axis acceleration while performing push-ups under controlled respiration cycles. (B) Baseline of z-axis acceleration.

FIG. 29 shows measured data during hammering nails, carrying boxes, and shoveling dirt. (A) Z-axis acceleration while hammering nails. (B) Z-axis acceleration while lifting, carrying, and placing a box. (C) Z-axis acceleration while shoveling dirt.

FIG. 30 shows motion artifacts from local movement. Local motion induced from movements of the neck. 3-axis acceleration data from the IMU1 on SN.

FIG. 31 shows feature classification with support vector machine (SVM). (A) Flowchart for event extraction using adapted thresholds from raw data. (B) Acceleration data recorded over 50 s with various activities that include tapping, coughing, laughing, and throat clearing. The blue line is the time series results of acceleration along the z-axis. The orange solid line is the envelope of the signal. The yellow line is the adapted threshold to detect specific features. The red dot is thcenter of the detected event. (C) Extracted samples after peak detection (1^{st} row), FFT (2^{nd} row), and spectrograms (3^{rd} row) of coughing, throat clearing, laughing and tapping. (D) Binary tree architecture design with SVM for classifying these activities. (E) Multiple SVM classification results. The 1^{st} classification result after SVM1 (left): negative values for throat clearing (yellow x). The 2^{nd} and 3^{rd} classification result after SVM2 and SVM3: classified tapping (brown triangle) after SVM2. Coughing (red circle) and laughing (blue inverted triangle) classification after SVM3.

FIG. 32 shows data set for developing the classifier. (A) Data including coughing, laughing, tapping, and throat clearing from 8 subjects. 10 data from each class in group 1 (SP1 to SP4) was used for classifier. (B) Coughing detection accuracy of each subject in group 1 and group 2.

FIG. 33 shows device temperature monitoring. (A) Experimental setup for monitoring the temperature of the device, with a focus on the battery. (B) Battery temperature measurement screen after 8 minutes of operation at room temperature. (C) Change in battery temperature over this time period.

FIGS. 34A-34C show schematically an electronic device according to various embodiments of the invention.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

The term “mechano-acoustic”, as used in the disclosure, refers to any sound, vibration or movement by a user that is detectable by an accelerometer or a gyroscope. Accordingly, accelerometers are preferably high frequency, three-axis accelerometers, capable of detecting a wide range of mechano-acoustic signals. Examples include respiration, swallowing, organ (lung, heart) movement, motion (scratching, exercise, and/or movement), talking, bowel activity, coughing, sneezing, and the like.

As used in the disclosure, the term “bidirectional wireless communication system” refers to onboard components of sensors, wireless controller and other electronic components that provides capability of receiving and sending signals using at least one communication protocol of near field communication (NFC), Wi-Fi/Internet, Bluetooth, Bluetooth low energy (BLE), and Cellular communication protocols for wireless communication. In this manner, an output may be provided to an external device, including a cloud-based device, personal portable device, or a caregiver's computer system. Similarly, a command may be sent to the sensor, such as by an external controller, which may or may not correspond to the external device. Machine learning algorithms may be employed to improve signal analysis and, in turn, command signals sent to the medical sensor, including a stimulator of the medical sensor for providing haptic signal to a user of the medical device useful in a therapy. More generally, these systems may be incorporated into a processor, such as a microprocessor located on-board or physically remote from the electronic device of the medical sensor. An example of the wireless controller is a near field communication (NFC) chip, including NFC chips. NFC is a radio technology enabling bi-directional short range wireless communication between devices. Another example of a wireless controller is a Bluetooth® chip, or a BLE system-on-chip (SoC), which enables devices to communicate via a standard radio frequency instead of through cables, wires or direct user action.

The term “flexibility” or “bendability”, as used in the disclosure, refers to the ability of a material, structure, device or device component to be deformed into a curved or bent shape without undergoing a transformation that introduces significant strain, such as strain characterizing the failure point of a material, structure, device or device component. In an exemplary embodiment, a flexible material, structure, device or device component may be deformed into a curved shape without introducing strain larger than or equal to 5%, for some applications larger than or equal to 1%, and for yet other applications larger than or equal to in strain-sensitive regions. A used herein, some, but not necessarily all, flexible structures are also stretchable. A variety of properties provide flexible structures (e.g., device components) of the invention, including materials properties such as a low modulus, bending stiffness and flexural rigidity; physical dimensions such as small average thickness (e.g., less than 100 microns, optionally less than 10 microns and optionally less than 1 micron) and device geometries such as thin film and open or mesh geometries.

The term “stretchable”, as used in the disclosure, refers to the ability of a material, structure, device or device component to be strained without undergoing fracture. In an exemplary embodiment, a stretchable material, structure, device or device component may undergo strain larger than 0.5% without fracturing, for some applications strain larger than 1% without fracturing and for yet other applications strain larger than 3% without fracturing. As used herein, many stretchable structures are also flexible. Some stretchable structures (e.g., device components) are engineered to be able to undergo compression, elongation and/or twisting so as to be able to deform without fracturing. Stretchable structures include thin film structures comprising stretchable materials, such as elastomers; bent structures capable of elongation, compression and/or twisting motion; and structures having an island-bridge geometry. Stretchable device components include structures having stretchable interconnects, such as stretchable electrical interconnects. As used herein, for embodiments where the devices are mounted directly to the skin, the devices may be characterized as stretchable, including stretchable and flexible so as to achieve good conformal contact with underlying skin, if desired. “Conformable” refers to a device, material or substrate which has a bending stiffness sufficiently low and elasticity sufficiently high to allow the device, material or substrate to adopt a desired contour profile, including a contour profile that may change over time, for example a contour profile allowing for conformal contact with a surface having a pattern of relief or recessed features, or. In certain embodiments, a desired contour profile is that of a tissue in a biological environment, for example skin or the epidermal layer.

The term “elastomer”, as used in the disclosure, refers to a polymeric material which can be stretched or deformed and return to its original shape without substantial permanent deformation. Elastomers commonly undergo substantially elastic deformations. Useful elastomers include those comprising polymers, copolymers, composite materials or mixtures of polymers and copolymers. Elastomeric layer refers to a layer comprising at least one elastomer.

Elastomeric layers may also include dopants and other non-elastomeric materials. Useful elastomers useful include, but are not limited to, thermoplastic elastomers, styrenic materials, olefenic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. In some embodiments, an elastomeric stamp comprises an elastomer. Exemplary elastomers include, but are not limited to silicon containing polymers such as polysiloxanes including poly(dimethyl siloxane) (i.e. PDMS and h-PDMS), poly(methyl siloxane), partially alkylated poly(methyl siloxane), poly(alkyl methyl siloxane) and poly(phenyl methyl siloxane), silicon modified elastomers, thermoplastic elastomers, styrenic materials, olefenic materials, polyolefin, polyurethane thermoplastic elastomers, polyamides, synthetic rubbers, polyisobutylene, poly(styrene-butadiene-styrene), polyurethanes, polychloroprene and silicones. In an embodiment, a flexible polymer is a flexible elastomer.

The term “encapsulate” or “encapsulation”, as used in the disclosure, refers to the orientation of one structure such that it is at least partially, and in some cases completely, surrounded by one or more other structures. “Partially encapsulated” refers to the orientation of one structure such that it is partially surrounded by one or more other structures. “Completely encapsulated” refers to the orientation of one structure such that it is completely surrounded by one or more other structures. The invention includes devices having partially or completely encapsulated electronic devices, device components and/or inorganic semiconductor components.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Soft, flexible, and wearable sensors offer the ability to continuously collect physiological parameters of relevance to human health. These systems offer the future ability to provide continuous biofeedback and even therapeutic benefit via on-board analytics and edge computing as we have previously disclosed. A key limitation of nearly all existing wearable sensors is motion artifact related deterioration of signal quality. This includes a lack of accurate sensing for heart rate, respiratory rate, body position, swallowing, talking, crying, or other respiratory signals in scenarios where the living subject is moving. Furthermore, core body temperature sensing remains a challenge for non-invasive skin mounted sensors. Thus, the ability to offer skin mounted continuous measurements of core body temperature would provide significant clinical utility.

One of the objectives of this invention is to provide a new class of wearable sensors that offers dramatically improved motion-resistant and ambient temperature resistant sensing enabled by novel device mechanics and design, and algorithms to subtract noises. This new class of wearable leverages differential measurement of outputs from sensors where one sensor is measuring physiological signals from the body and related ambient and gross body motion signals, and another sensor is measuring at least the related ambient and gross body motion signals allow for effective elimination of noise, e.g., related ambient and gross body motion signals, during rest and motion.

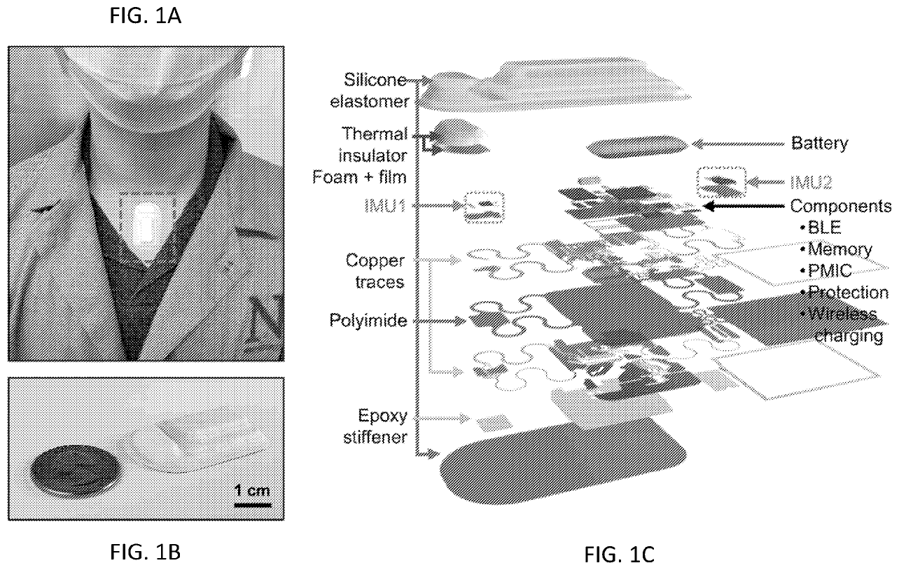

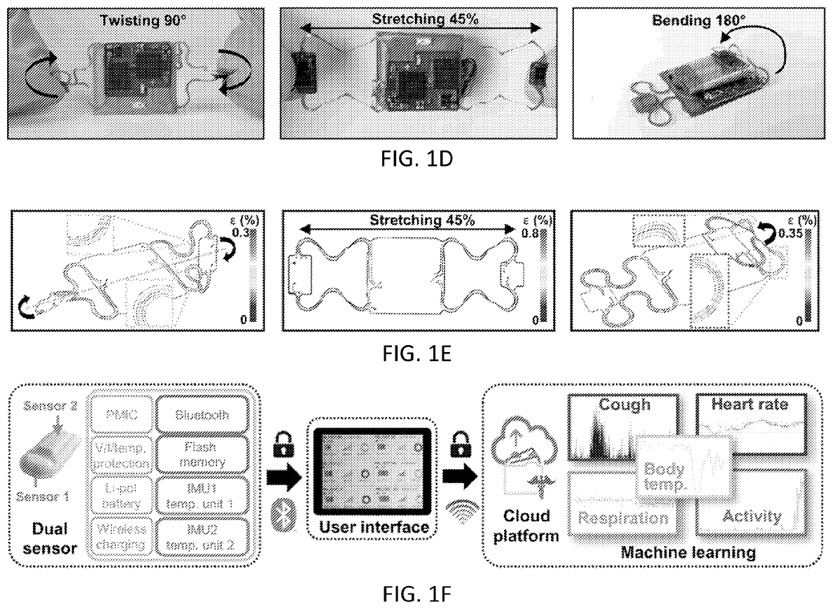

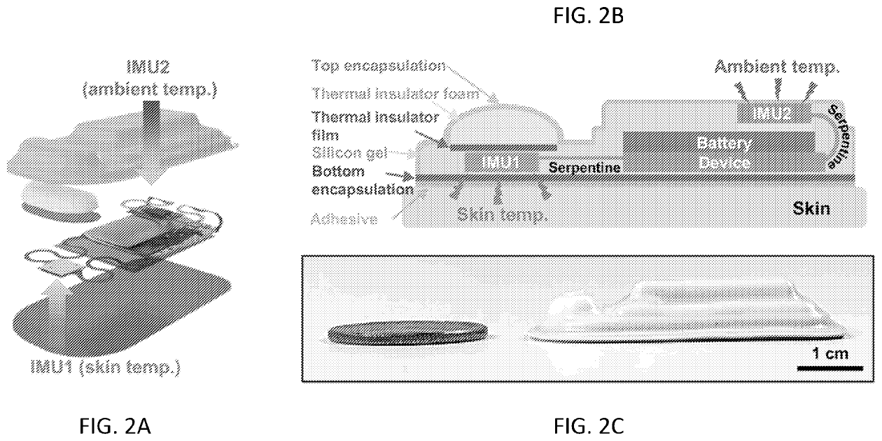

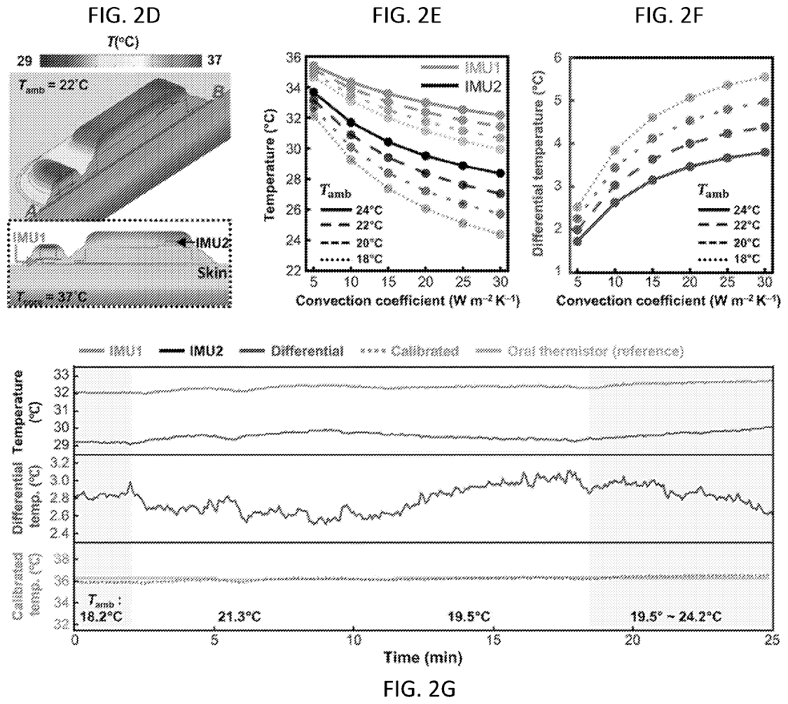

In certain aspects, the invention relates to an electronic device for measuring physiological parameters of a living subject. As shown in FIGS. 1C and 2B, the electronic device in one embodiment includes at least a first inertial measurement unit (IMU) and a second IMU, the first IMU and the second IMU are time-synchronized to and spatially and mechanically separated from each other; and a microcontroller unit (MCU) electronically coupled to the first IMU and the second IMU for processing of data streams from the first IMU and the second IMU. In certain embodiments, the first IMU is configured to measure data including a first signal related to a physiological signal of the living subject and a second signal, and the second IMU is configured to measure data including at least the second signal. The first signal measured by the first IMU has a signal strength greater than that the second signal measured by the first IMU.

In certain embodiments, the data measured by the first IMU and the second IMU are processed such that subtraction of the second signal measured by the second sensor from the second signal measured by the first sensor results in a stronger first signal that is a signal of interest.

In certain embodiments, the second signal is related to at least one of ambient, motion and vibration.

In certain embodiments, the data measured by the second IMU includes the first signal and the second signal.

In certain embodiments, a signal-to-noise ratio (SNR) of a signal measured by the first IMU and the second IMU together is lower than a first SNR of a signal measured by the first IMU individually, or a second SNR of a signal measured by the second IMU individually.

In certain embodiments, both of the first IMU and the second IMU are operably in mechanical communication with the skin of the living subject.

In certain embodiments, one of the first IMU and the second IMU is operably in directly mechanical communication with the skin of the living subject for sensing physiological signals of the body, while the other of the first IMU and the second IMU is operably in indirectly mechanical communication with the skin of the living subject.

In certain embodiments, the first IMU and the second IMU are operably in directly mechanical communication with the skin of the living subject.

In certain embodiments, one of the first IMU and the second IMU is separated from the rest of rigid components of the electronic device.

In certain embodiments, the electronic device also comprises at least first and second thermal sensing units, wherein one of the first and second thermal sensing units is thermally isolated from an ambient environment and configured to measure a body temperature of the living subject, and the other of the first and second thermal sensing units is configured to measure the ambient temperature.

In certain embodiments, each of the first and second thermal sensing units is embedded in a respective one of the first and second IMUs.

In certain embodiments, the electronic device is configured to measure a range of physiological information from activity of a cardiopulmonary system and movements of a core body to a diverse collection of processes across thoracic cavity, esophagus, pharynx, and oral cavity related to respiration, speech, swallowing, wheezing, coughing, and sneezing.

In certain embodiments, the electronic device is configured to separate signals associated with the cardiopulmonary system and related processes from those due to body movements.

In certain embodiments, the electronic device is configured to spatiotemporally map movements of the skin at this region of the anatomy onto which the electronic device is attached during cardiac and respiratory activities.

In certain embodiments, the electronic device is configured to continuously measure temperature, heart rate (HR), respiratory rate (RR), activity level, and body orientation, across a range of vigorous activities and conditions.

In certain embodiments, the electronic device is configured to monitor key symptoms of a patient with COVID-19 infection to track progress of recovery and response to therapies in hospital and/or home.

In certain embodiments, the electronic device is configured to measure any of respiratory or motion related digital biomarkers associated with coughing, swallowing, and/or specific motion related activities.

In certain embodiments, the electronic device is configured to assess coughing when the living subject is moving or immobile, and/or to measure muscle motion, when the living subject is moving.

In certain embodiments, the electronic device further comprises a bidirectional wireless communication system electronically coupled to the electronic device and configured to send an output signal from the electronic device to an external device.

In certain embodiments, the external device is a mobile device, a computer, or a cloud service.

In certain embodiments, the bidirectional wireless communication system is further configured to deliver commands from the external device to the electronic device.

In certain embodiments, the bidirectional wireless communication system comprises a controller that utilizes at least one of near field communication (NFC), Wi-Fi/Internet, Bluetooth, Bluetooth low energy (BLE), and cellular communication protocols for wireless communication.

In certain embodiments, the electronic device further comprises a customized app with a user interface deployed in the external device to allow a user to configure and operate the electronic device for data collection, data transfer, data storage and analysis, wireless charging, and monitoring of user's conditions.

In certain embodiments, the customized app is configured to allow time-synchronized operation of a plurality of the electronic devices simultaneously.

In certain embodiments, the electronic device further comprises a power module coupled to the first IMU, the second IMU and the MCU for providing power thereto.

In certain embodiments, the power module comprises at least one battery for providing the power. In certain embodiments, the battery is a rechargeable battery.

In certain embodiments, the power module further comprises a wireless charging module for wirelessly charging the rechargeable battery.

In certain embodiments, the power module further comprises a failure prevention element including a short-circuit protection component or a circuit to avoid battery malfunction.

In certain embodiments, the second IMU is placed in a manner that it bends and folds over the battery.

In certain embodiments, the electronic device further comprises a flexible printed circuit board (fPCB) having flexible and stretchable interconnects electrically connecting to electronic components including the first IMU, the second IMU and the MCU and the power module.

In certain embodiments, the electronic device further comprises an elastomeric encapsulation layer at least partially surrounding the electronic components and the flexible and stretchable interconnects to form a tissue-facing surface attached to the living subject and an environment-facing surface, wherein the tissue-facing surface is configured to conform to a skin surface of the living subject.

In certain embodiments, the encapsulation layer is formed of a flame retardant material.

In certain embodiments, the elastomeric encapsulation layer is a waterproof and biocompatible silicone enclosure.

In certain embodiments, the electronic device further comprises a biocompatible hydrogel adhesive for attaching the electronic device on the respective region of the living subject, wherein the biocompatible hydrogel adhesive is adapted such that signals from the living subject are operably conductible to the first IMU and the second IMU.

In certain embodiments, the electronic device is flexible and conformable to the skin with a specific geometrical polarity for mounting in an anatomical location of interest of the living subject.

In certain embodiments, the electronic device is wearable, tissue mountable or in mechanical communication or direct mechanical communication with the skin of the living subject. As used herein mechanical communication refers to the ability for the sensors to interface directly or indirectly with the skin or other tissue in a conformable, flexible, and direct manner (e.g., there is no air gap) which in some embodiments allows for deeper insights and better sensing with less motion artifact compared to accelerometers strapped to the body (wrists or chest).

In certain embodiments, the electronic device is twistable stretchable, and/or bendable.

Various embodiments of the present technology include a soft, conformal, stretchable class of device configured specifically for mechano-acoustic recording from the skin, capable of being used on nearly any part of the body, in forms that maximize detectable signals and allow for multimodal operation, such as electrophysiological recording, and neurocognitive interaction.

Another aspect of the invention provides an electronic device comprising a sensor network comprising a plurality of sensor units operably deployed on a skin of the living subject, the plurality of sensor units being time-synchronized to and spatially and mechanically separated from each other; and an MCU electronically coupled to the plurality of sensor units for processing of data streams from the plurality of sensor units.

In certain embodiments, the plurality of sensor units are configured to measure a same physiological parameter, or different physiological parameters.

In certain embodiments, each of the plurality of sensor units comprises at least a first sensor and the second sensor time-synchronized to and spatially and mechanically separated from each other.

In certain embodiments, for each sensor unit, the first sensor is configured to measure data including a first signal related to a physiological signal of the living subject and a second signal, and the second sensor is configured to measure data including at least the second signal. The first signal measured by the first sensor has a signal strength greater than that the second signal measured by the first sensor.

In certain embodiments, the data measured by the first sensor and the second sensor of said sensor unit are processed such that subtraction of the second signal measured by the second sensor from the second signal measured by the first sensor results in a stronger first signal that is a signal of interest

In certain embodiments, the second signal is related to at least one of ambient, motion and vibration.

In certain embodiments, each of the first sensor and the second sensor comprises the IMU.

In certain embodiments, the electronic device further comprises a plurality of thermal sensing units.

In certain embodiments, the MCU operably receives inputs from synchronized outputs of a plurality of thermal sensor units with at least one thermal sensing unit for the ambient environment and at least one thermal sensing unit in direct thermal communication from the body isolated thermally from the ambient environment with in-sensor thermally isolating materials.

In certain embodiments, the electronic device is configured to automatically switch operation modes, the operation modes include at least a first mode when the living subject is at rest, and a second modes when the living subject is in a high motion.

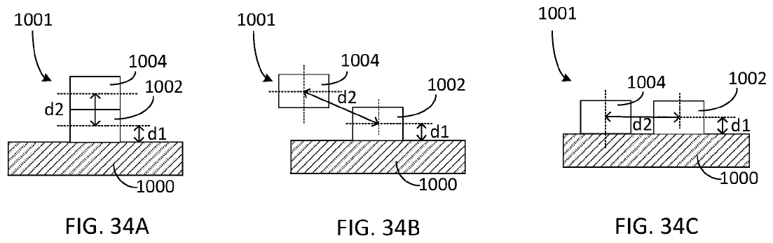

Yet another aspect of the invention provides an electronic device 1001 for measuring physiological parameters of a living subject 1000, as shown in FIGS. 34A-34C. The electronic device 1001 comprises a first sensor 1002 adapted for detecting a first group of data related to the living subject 1000 and a second group of data that is different from the first group of data; and a second sensor 1004 for detecting a third group of data that is substantially similar to the second group of data. In operation, the first sensor 1002 and the second sensor 1004 are time-synchronized to allow the third group of data from the second sensor to be used to substantially cancel out the second group of data from the first sensor.

In certain embodiments, the first sensor and the second sensor are spatially and mechanically separated from each other.

In certain embodiments, the separation of the first sensor and the second sensor is greater than zero and less than a predetermined distance.

In certain embodiments, each of the first sensor and the second sensor comprises an IMU, a thermal sensor, and/or a pressure sensor.

In certain embodiments, the first group of data is physiological signals of the living subject, and the second group of data is signals related to ambient, motion and/or vibration at the first sensor.

In certain embodiments, the third group of data is signals related to ambient, motion and/or vibration at the second sensor.

In certain embodiments, both of the first sensor and second sensor are operably in mechanical communication with the skin of the living subject.

In certain embodiments, the first sensor is operably in directly mechanical communication with the skin of the living subject for sensing physiological signals from the body, and the second sensor is operably in indirectly mechanical communication with the skin of the living subject.

In certain embodiments, the first sensor and the second sensor are operably in directly mechanical communication with the skin of the living subject for sensing physiological signals from the body to assess pulse transit time.

In certain embodiments, the electronic device is flexible and conformable to the skin with a specific geometrical polarity for mounting in an anatomical location of interest of the living subject.

A further aspect of the invention provides an electronic device 1001 for measuring physiological parameters of a living subject 1000, as shown in FIGS. 34A-34C. The electronic device 1001 comprises a first sensor 1002 adapted for detecting a first group of data related to the living subject 1000 and a second group of data that is different from the first group of data; and a second sensor 1004 for detecting a third group of data that is substantially similar to the second group of data, wherein in operation, the first sensor 1002 is positioned such that there is a first distance d1 between a center of the first sensor 1002 and an area of the living subject 1000 where physiological signals of the living subject 1000 are measurable; the second sensor 1004 is positioned such that there is a second distance d2 between a center of the second sensor 1004 and the center of the first sensor 1002, wherein the second distance d2 is greater than zero and less than a predetermined distance.

In one embodiment, the second sensor 1004 is positioned over the first sensor 1002, as shown in FIG. 34A.

In one embodiment, the second sensor 1004 is positioned away from the first sensor 1002, as shown in FIGS. 34B-34C.

In certain embodiments, measurements of physiological parameters can be derived from mechano-acoustic signals from the human body of heart rate, respiratory rate, body position, swallow count, cry time, talk time, singing, coughing, and differential motion of specific body parts when the sensor is mounted across an anatomical boundary (e.g., trunk motion in relation to the head, hand motion in relation to the wrist, lower leg motion in relation to the knee), and other respiratory signals at rest and during motion.

In certain embodiments, the derivation of these physiological resistant to motion allows for applicability across a wide range of medical specialties and acuity ranging from critical care, general medicine care, ambulatory medicine, rehabilitation, and consumer health particularly in high motion scenarios.

In certain embodiments, the core body sensing is resistant to ambient temperature fluctuations and clothing.

In certain embodiments, the single MCU receives inputs from synchronized outputs of a plurality of IMU sensors where an individual IMU sensor is in differential mechanical communication with the body.

In certain embodiments, the single MCU receives inputs from synchronized outputs of a plurality of thermal sensors with at least one thermal sensor for the ambient environment and at least one thermal sensor in direct thermal communication from the body isolated thermally from the ambient with in-sensor thermally isolating materials.

In certain embodiments, the novel mechanics of the sensor/device allows for twisting, stretching, bending to enable a low profile design and thermal/mechanical isolation of various sensing elements in the device.

In certain embodiments, the novel mechanics of the sensor/device enables a physical separation of rigid components of the device and the sensing element enabling mounting in unique anatomical locations for high data fidelity. Further advantages include the ability to obscure the sensor from sight to reduce patient stigma. This represents an umbilical functionality to allow for discrete sensing in sensitive locations with the body of the sensor is mounted in a location easier to obscure with clothing.

In certain embodiments, thermal isolating materials and layers that allow for improved thermal sensing of core body temperature that is resistant to ambient temperature fluctuations. In certain embodiments, the sensor/device is configured such that the modes of operation in situations of high motion allows for automated switching to motion resistant outputs. For example, the preferred measurement of heart rate may be ECG at rest—however, a patient maybe in a situation where they are actively moving. In this instance, the sensor can start actively interrogating the dual IMUS for heart rate derivation where ECG based heart rate is not dependable.

In certain embodiments, the sensor/device has ability to toggle or activate dual sensing functionality in situations of high motion to improve accuracy but conserve power in settings of rest.

The techniques introduced here can be embodied as special purpose hardware (e.g. circuitry) as programmable circuitry appropriately programmed with software and/or firmware, or as a combination of special-purpose and programmable circuitry. Hence, embodiment may include a machine-readable medium having stored thereon instructions which may be used to program a computer (or other electronic devices) to perform a process. The machine-readable medium may include, but is not limited to, floppy diskettes, optical disks, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions.

These and other aspects of the present invention are further described below. Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example

### Differential Cardiopulmonary Monitoring System for Artifact-Canceled Physiological Tracking of Athletes, Workers, and COVID-19 Patients

Soft, skin-integrated electronic sensors can provide continuous measurements of diverse physiological parameters, with broad relevance to the future of human health care. Motion artifacts can, however, corrupt the recorded signals, particularly those associated with mechanical signatures of cardiopulmonary processes. Design strategies introduced herein address this limitation through differential operation of a matched, time-synchronized pair of high-bandwidth accelerometers located on parts of the anatomy that exhibit strong spatial gradients in motion characteristics. When mounted at a location that spans the suprasternal notch and the sternal manubrium, these dual-sensing devices allow measurements of heart rate and sounds, respiratory activities, body temperature, body orientation, and activity level, along with swallowing, coughing, talking, and related processes, without sensitivity to ambient conditions during routine daily activities, vigorous exercises, intense manual labor, and even swimming. Deployments on patients with COVID-19 allow clinical-grade ambulatory monitoring of the key symptoms of the disease even during rehabilitation protocols.

Specifically, the exemplary work exploits a pair of time-synchronized, high-bandwidth accelerometers (inertial measurement units (IMUs)) at opposite ends of a skin-interfaced device that locates one of the IMUs at the suprasternal notch (SN) and the other at the sternal manubrium (SM). Differences in movements of the skin associated with cardiac and respiratory activity between these regions lead to differences in signals captured by these IMUs. By contrast, overall movements of the neck and the core of the body produce nearly identical responses. As a consequence, simple differential measurements cleanly eliminate common mode features, thereby separating signals associated with cardiopulmonary and related processes from those due to body movements. An additional benefit of this architecture is that temperature sensors integrated in these IMUs can be used in a similar differential manner to yield estimates of core body temperature, largely independent of the ambient. Here, careful choices in thermal aspects of the device layout, rather than intrinsic anatomical gradients, produce the necessary differential responses.

The following sections present (i) designs of automated devices that incorporate matched pairs of high-bandwidth IMUs with optimized soft mechanics for high measurement sensitivity and accurate time synchronization across the SN and SM; (ii) results of spatiotemporal mapping of movements of the skin at this region of the anatomy during cardiac and respiratory activity; (iii) examples of modeling and design approaches for exploiting these IMUs in dual temperature sensing of core body temperature, with minimal influence of the thermal ambient; (iv) demonstrations of continuous, differential measurements of temperature, HR, and respiratory rate (RR) across a range of vigorous activities and conditions, with benchmarking against the most accurate commercial sensors; and (v) illustrations of the use on patients recovering from COVID-19 infections to track key symptoms of the disease even during intense physical rehabilitation protocols.

### Results

### Design and Characterization

The platform exploits a thin, flexible printed circuit board (fPCB) in an open architecture, with an elastomeric encapsulation structure that completely seals the system to physically isolate the electronics from the environment and to facilitate sterilization for reuse. The layouts yield soft mechanical characteristics for comfortable mounting on the skin, even at sensitive regions of the body. A touch-free docking interrogator supports wireless charging and initiates data downloads in an automated fashion to eliminate user burden.

FIG. 1A shows a device mounted on the base of the neck, positioned to span the SN and SM. This unique anatomical location allows for measurements of a rich range of biophysical information, from activity of the cardiopulmonary system and movements of the core body to a diverse collection of processes across the thoracic cavity, esophagus, pharynx, and oral cavity related to respiration, speech, swallowing, wheezing, coughing, and sneezing.

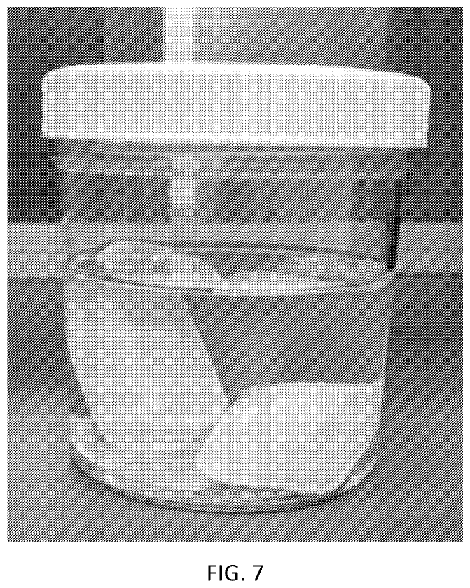

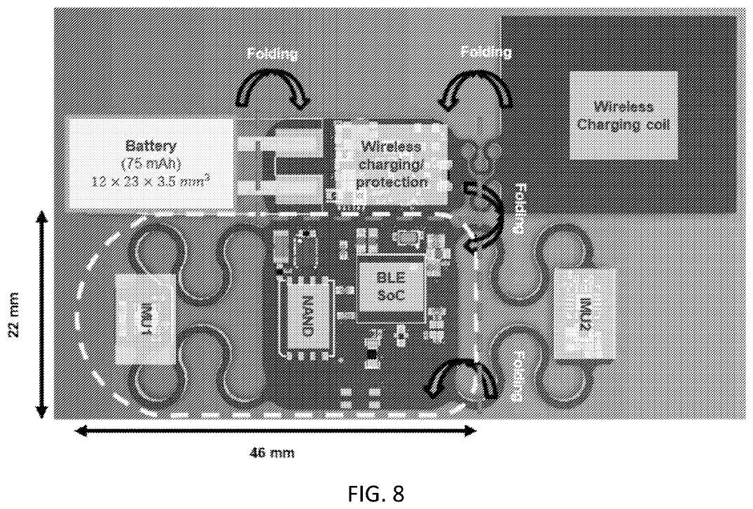

FIG. 1B presents an exploded-view schematic illustration of the soft enclosure and the fPCB with passive/active chip-scale components. Top and bottom encapsulating films of a silicone elastomer (thickness, 0.3 mm; Silbione RTV 4420) mechanically isolate the active parts of the systems in a sealed enclosure that allows operation even when submerged in water or exposed to sweat. The design is also compatible with U.S. Centers for Disease Control and Prevention guidelines for cleaning and disinfecting using 70% alcohol solutions (FIG. 7). The fPCB exploits a copper (12 μm)-polyimide (PI; 25 μm)-copper (12 μm) laminate (DuPont, AP7164R) patterned to define conductive traces with widths of 80 and 150 μm. The layout (FIG. 8) includes separate islands for the circuit components (main body), each of the two IMUs (IMU1 and IMU2), and a wireless charging coil. Serpentine-shaped traces interconnect these islands to mechanically decouple the IMUs from one another, as necessary in precision, differential measurements of MA signals at the surface of the skin. Specifically, two pairs of narrow, filamentary serpentine structures electrically connect the IMUs to the main body in a manner that minimizes mechanical constraints. The fPCB also includes multiple zones to allow for static bending during an assembly process that folds the system into a compact configuration. The image in FIG. 1C shows the overall size relative to a U.S. quarter (diameter, 24.26 mm). The dimensions of the encapsulated device are 46 mm by 22 mm; its thickness is less than 9 mm, and its weight is less than 6.35 g.

Experimental studies, as shown in FIG. 1D, and finite element analysis (FEA) computations, as shown in FIG. 1E, confirm that the strains in the copper of the serpentine interconnects remain below fracture limits (6=1%) throughout the assembly process and during operation under different types of external loads: 90° torsional (the left frame in FIGS. 1D-1E), 45% tensile (the middle frame in FIGS. 1D-1E), and 180° bending (the right frame in FIGS. 1D-1E) deformations. This soft, stretchable design can accommodate deformations of the skin at the SN without fatigue or fracture and with minimal irritation and discomfort. Stiffening layers beneath critical regions of the platform reduce the probability for bending/stretching-induced damage of the solder joints. The result is a mechanically robust platform that enables high sensitivity measurements of both subtle motions of the skin and full body kinematics across a broad range of frequencies. Descriptions of additional design features, including those related to thermal measurements, appear subsequently.

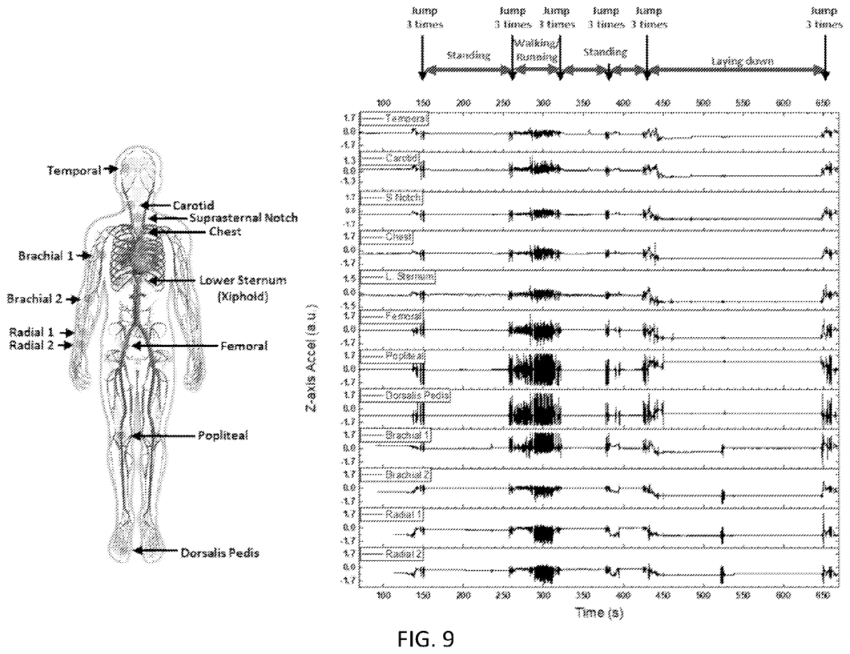

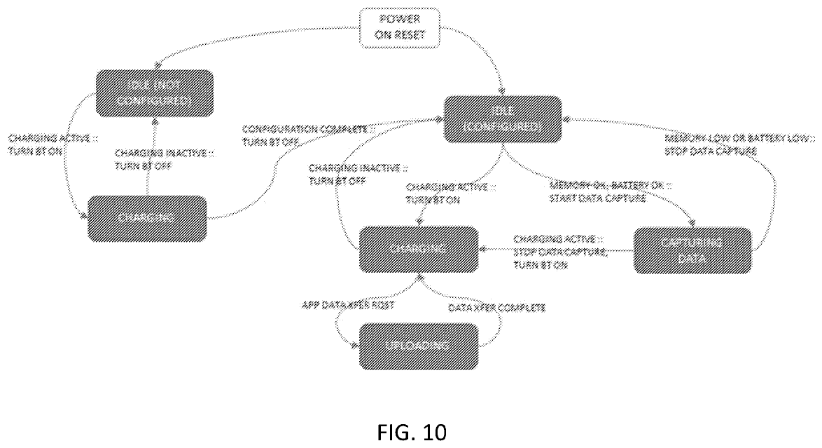

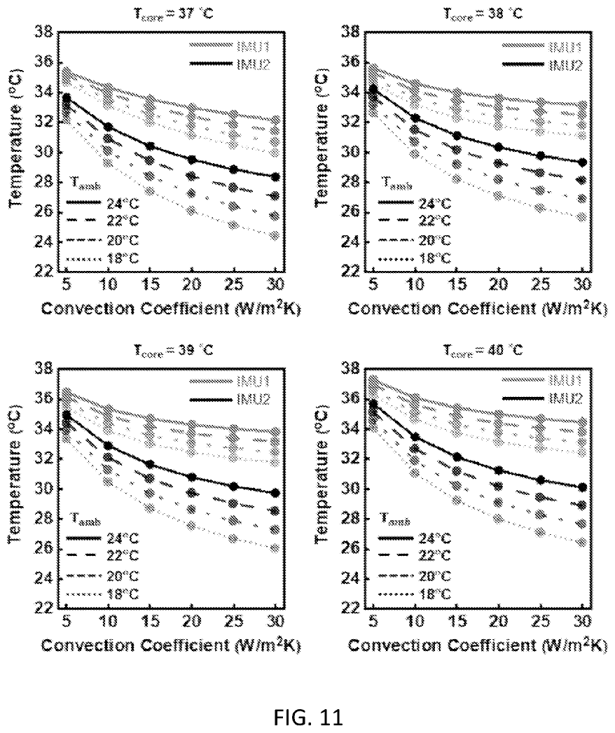

The block diagram in FIG. 1F summarizes the overall system operation. The three main components include the device, a tablet with a customized app as a user interface, and a cloud platform for data storage and analytics. The device uses a BLE SoC (Bluetooth Low Energy System on a Chip) (Nordic Semiconductor, nRF52840), a PMIC (power management integrated circuit) (Texas Instruments, BQ25120), a 4-gigabit NAND flash memory (Micron, MT29F4G01), and two identical IMUs, each with an embedded temperature sensing unit (STMicroelectronics, LSM6DSL). Wireless charging involves voltage and current protection as support for a 75-mA·hour lithium polymer battery. The user interface allows time-synchronized operation of up to 12 devices, simultaneously. Although not explored in the following experiments in this study, this feature supports monitoring of social interactions and/or capture of MA signals at multiple body locations (FIG. 9). FIG. 10 describes the state diagram of the system, to illustrate behaviors before and after configuration, followed by deployment on a subject. The diagram also shows operation during data collection, charging, and data transfer.

Full automation of the key operational steps minimizes user burden, of particular importance for use with patients with COVID-19, as described subsequently. The user simply mounts the device during use and places it on the wireless charging platform when removed. The sensor continuously stores data from both accelerometers onto the internal memory module when not on the charging platform; when on this platform, the device charges and simultaneously streams data to a user interface device via Bluetooth protocols. The user interface then passes data to a cloud hub for signal processing to extract various physiological information, including cough count, RR, HR, activity level, body orientation, and calibrated body temperature. The cloud hub is HIPAA (Health Insurance Portability and Accountability Act) compliant, and the interface application uses HTTPS transport layer security (TLS 1.2) with an algorithm for encryption/decryption for the application programming interface and a standard for in-storage encryption (AES-256).

Core Body Temperature Estimation with Dual Temperature Sensing

The simplest consequence of the dual-sensing architecture is in temperature measurements that approximate the temperature of the skin (Tₛₖᵢₙ), largely unperturbed by the ambient (T_{amb}), following schemes described previously in other contexts. Here, sensors embedded in IMU1 and IMU2, in a configuration illustrated in FIG. 2A, yield temperatures with repeatability of 0.004° C. every 5 s (adjustable up to a 52-Hz sampling rate). IMU1 rests directly adjacent to the skin, separated only by the thin bottom encapsulation layer (0.3-mm-thick silicone elastomer). A 6-mm-thick thermally insulating foam (polyurethane mixture) with a metallic film (12-μm-thick aluminized polyethylene) minimizes coupling to the environment via convection, conduction, and radiation. The temperature at IMU1 depends strongly on the core body temperature, modulated by the effective thermal properties of the tissues and the ambient conditions. IMU2 resides on the outward-facing side of the device, with only the top encapsulation layer above, to maximize and coupling to the environment (FIGS. 2B-2C). The multiple underlying layers, including the adhesive film, bottom encapsulation, fPCB, battery, and thermal insulating foam limit heat transfer from the skin to IMU2.

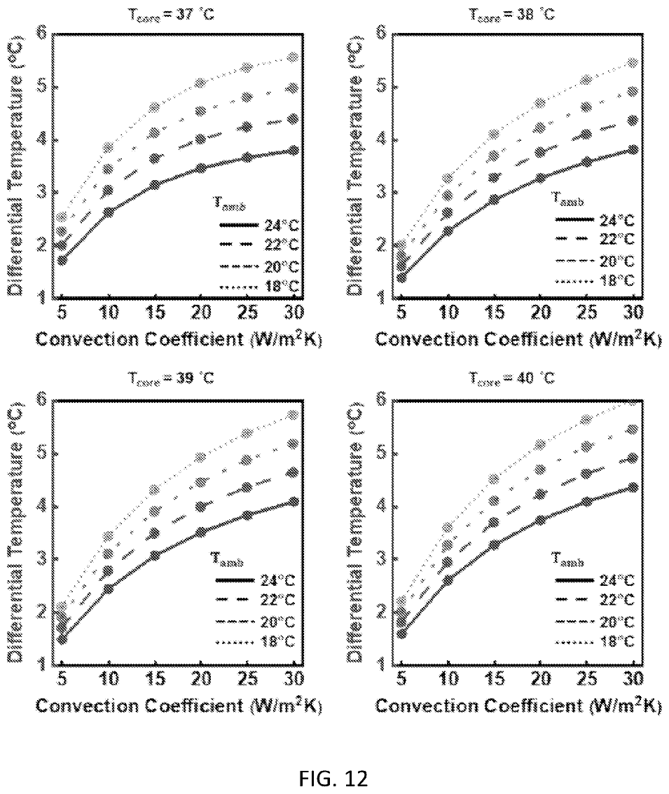

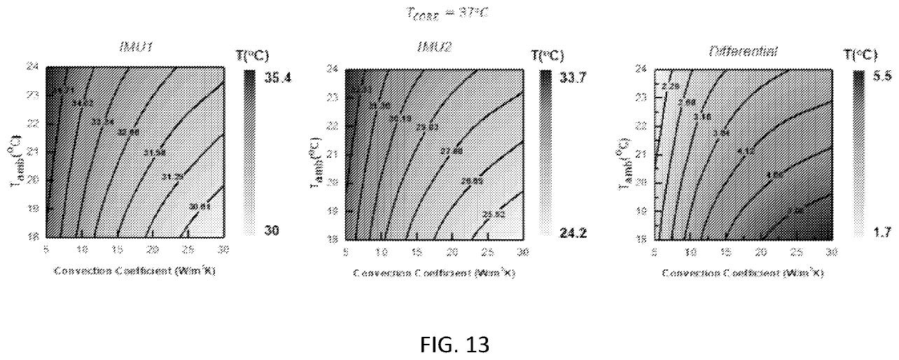

Transient heat transfer analysis associated with three-dimensional (3D) thermal conduction and natural convection quantifies these effects. The boundary conditions include a constant temperature at the bottom surface of the tissue layer (T_{core}) and convective coupling to the ambient air at the free surfaces (T_{amb}). The parameters include the room temperature, T_{amb}=18° to 24° C., and the convection coefficient, h=5 to 30 W m⁻² K⁻¹. FIG. 2D highlights temperature distributions across the skin and regions surrounding the device for T_{core}=37° C., T_{amb}=22° C., and h=10 W m⁻² K⁻¹. The temperature profile along the AB cross section shows results inside the device with T_{core}=37° C. (FIG. 2E) and with T_{core}=38° to 40° C. (FIG. 11). FIG. 2F summarizes the differences in temperature between the two IMUS (T_{diff}) for T_{core}=37° C. As might be expected, reducing T_{amb} and/or increasing h increases T_{diff}. As specific examples, for h=5 W m⁻² K⁻¹, T_{diff} 1.72° C. when T_{amb}=24° C. and T_{diff}=2.52° C. when T_{amb}=18° C. In addition, for h=30 W m⁻² K⁻¹, T_{diff}=3.80° C. when T_{amb}=24° C., but T_{diff}=5.55° C. when T_{amb}=18° C. In the same way, FIG. 12 shows temperature differences under the condition of T_{core}=38° to 40° C., with associated temperature distributions in FIG. 13. The analysis also quantifies the effects of changes in the ambient temperature, the core body temperature, the convection coefficient, and other key parameters. Measurements of differential temperature together with subject-specific thermal models yield robust estimates of core body temperature.

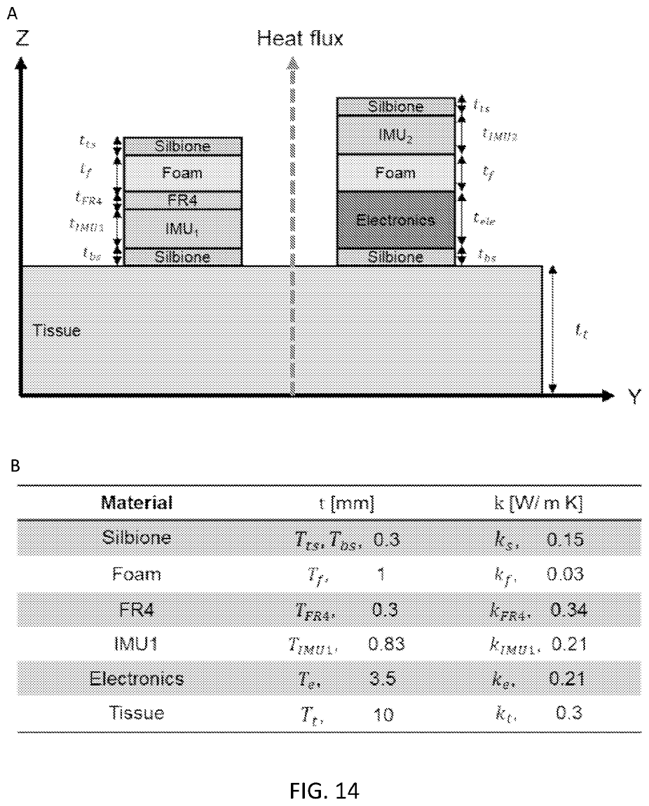

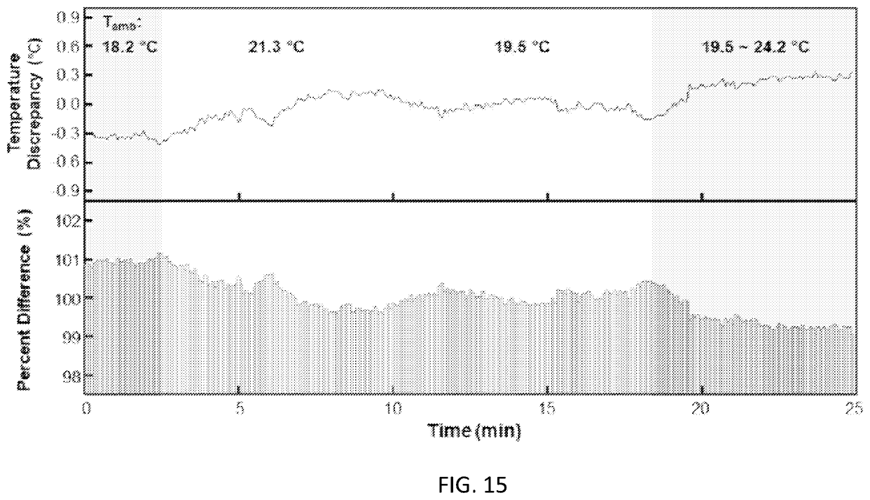

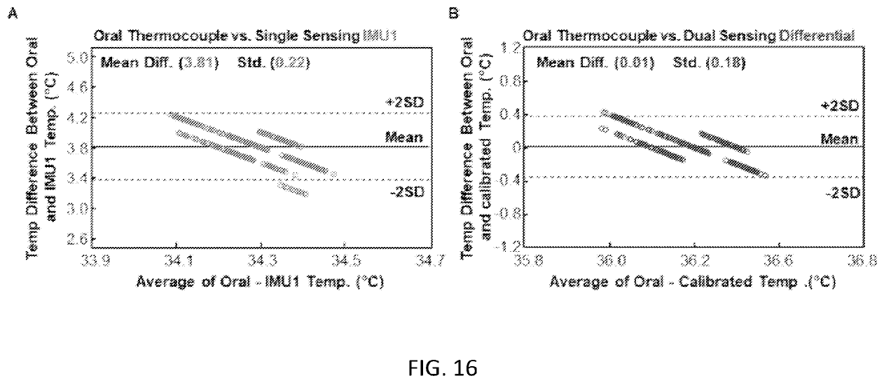

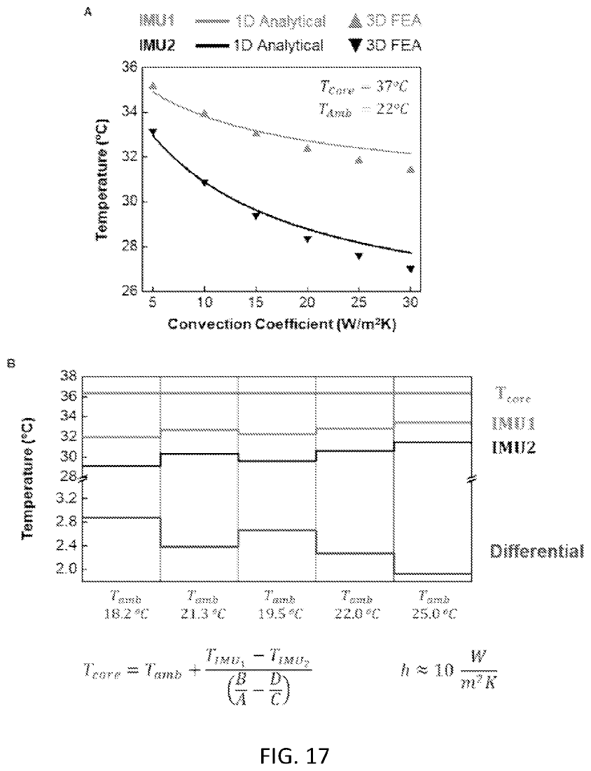

A simple demonstration involves a subject wearing a device in an environment with an ambient temperature of 18.2° C., then moving between areas with temperatures of 21.3° and 19.5° C. every 3 to 8 min, and lastly remaining in place as the ambient temperature rises from 19.5° to 24.2° C. for 7 min. The results for temperatures recorded from IMU1 and IMU2 appear in the top graph in FIG. 2G. The middle graph shows the differential temperature. A subject-specific model converts these temperature measurements into estimates of core body temperature (third row in FIG. 2G), determined by eq. (17) in the “1-D analytical model for the thermal characteristics” section in Materials and Methods, T_{core}=T_{amb}+(T_{IMU1}−T_{IMU2})÷(B/A−D/C), where T_{amb} is the ambient temperature inferred from IMU2, T_{IMU1} is the temperature from IMU1, T_{IMU2} is the temperature from IMU2, and (B/A−D/C) is a quantity that depends on the thickness and heat transfer coefficients of the skin and the various material layers of the device. Details of the structures, values, equations, and the modeling approaches appear in FIG. 14 (see the 1-D analytical model for the thermal characteristics section in Materials and Methods). A thermocouple placed under the tongue (green curve in the bottom graph of FIG. 2G) yields reference values that approximate the core body temperature. FIG. 15 compares the results to the core temperature estimated from measurements at IMU1 and IMU2. The differences remain less than ˜0.5° C. across ambient temperatures from 19.5° to 24.2° C. FIG. 16 shows Bland-Altman plots of the data. Sensing with only IMU1 (red) yields a mean difference of 3.81° C. and an SD of compared to the oral measurement; the dual-sensing approach (blue) yields a mean difference of 0.01° C. and an SD of 0.18° C. A 1D heat transfer model for analytics and 3D FEA model of the temperature dynamics (FIG. 17) can capture essential aspects of these demonstrations. The analytical and 3D FEA results agree well over the range of different ambient temperature scenarios with relevant heat convection coefficients and the core temperature (36.3° C.), similar to the experimental results in FIGS. 2G and 17 (B).

Dual Sensing from the SN and the SM

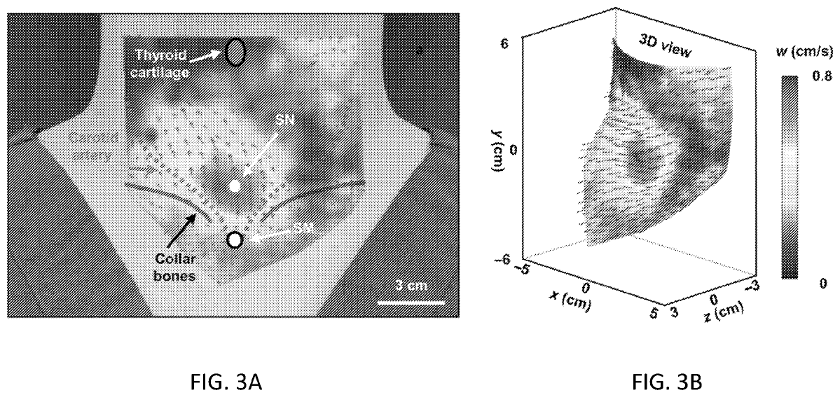

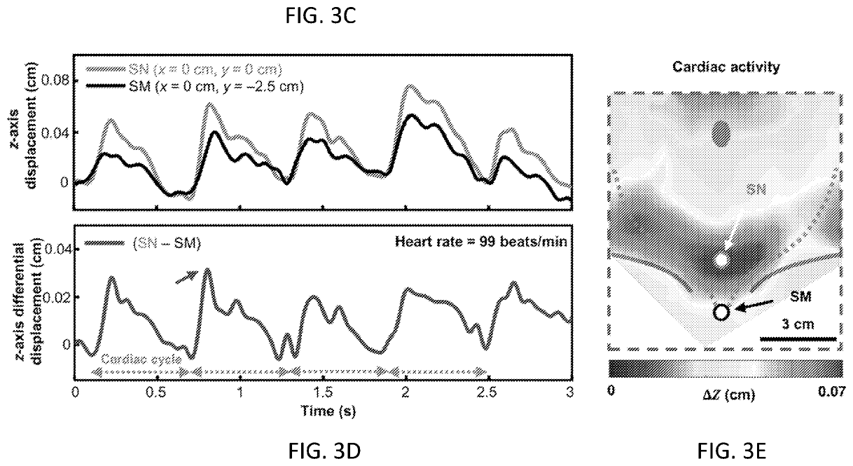

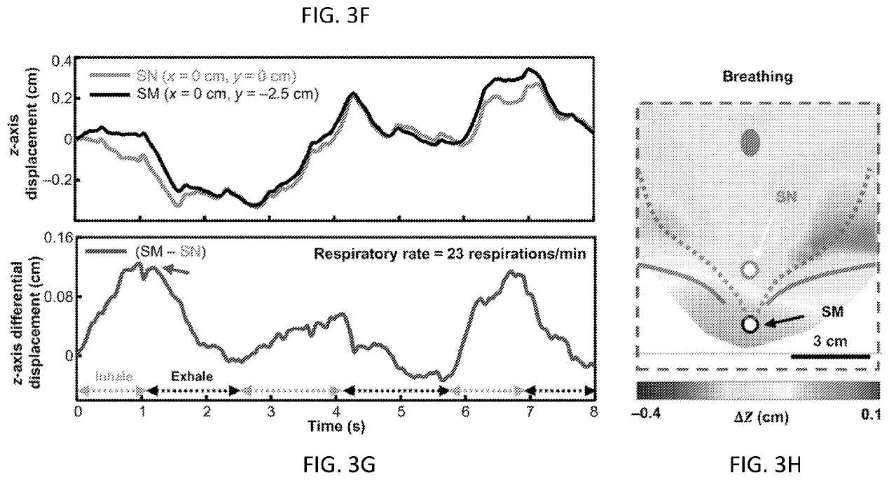

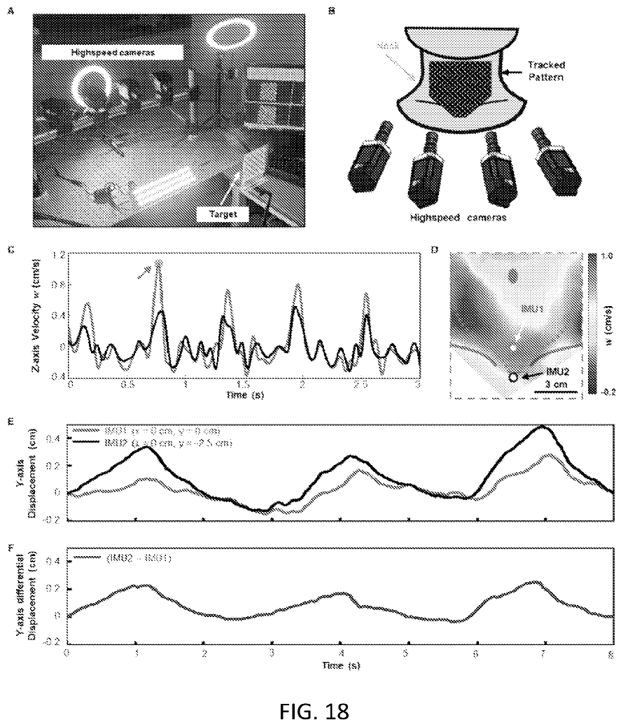

Dual temperature sensing relies critically on design choices that yield different levels of sensitivity to temperatures of the body and the ambient for IMU1 and IMU2. For dual MA sensing, differential responses arise mainly from spatial gradients in motions across the mounting location, specifically those from the SN, the location of IMU1 and from the SM, the location of IMU2 (2.5 cm below the IMU1). Spatiotemporal maps of motions of this region of the anatomy determined by 3D particle tracking velocimetry (3D-PTV) provide quantitative insights into differential motions associated with respiratory and cardiac activities at these two locations and adjacent regions. 3D-PTV relies on optical techniques to track the Lagrangian paths of fiducial marks on the skin, in 3D using stereoscopic imaging, in a way that recapitulates the point-measurement modality of the IMUs. Here, 3D-PTV can capture the essence of dual sensing from the SN and SM by recording from four time-synchronized, high-speed cameras, each at a frame rate of 200 frames per second (fps) (FIG. 18), and track motions across the neck, including regions of the SN and SM (FIG. 3A). Displacement and vector contour fields follow from interpolation of fiducials at each frame based on Delaunay triangulation (FIG. 3B). The results featured here correspond to representative velocities measured near the peak of a cardiac cycle, relative to those between cycles, for a subject at rest. The results reveal notable differences between motions at the SN and SM, as the basis for differential detection.

FIGS. 3C-3E show additional detail, corresponding to z-axis displacement profiles through several cardiac cycles during a breath hold after a brief period of exercise (20 push-ups). The peak displacements at the SN are −50% larger than those at the SM, as shown in FIG. 3C. By contrast, the displacements associated with body motions are nearly identical (as expected, but not explicitly shown here), thereby allowing for efficient subtraction. The differential result appears in FIG. 3D. FIG. 3E shows a color contour plot of z-axis displacements at the peak of the cardiac cycle highlighted by the arrow in FIGS. 3D and 18 (C-D).

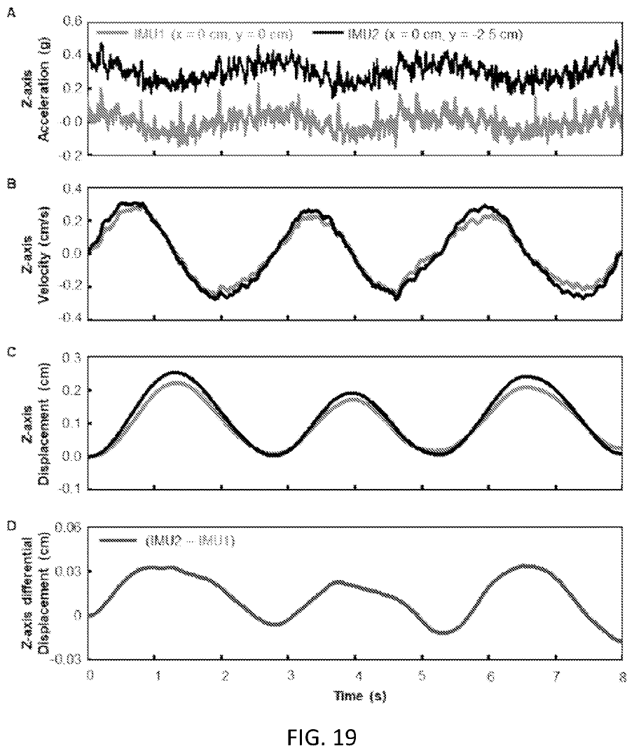

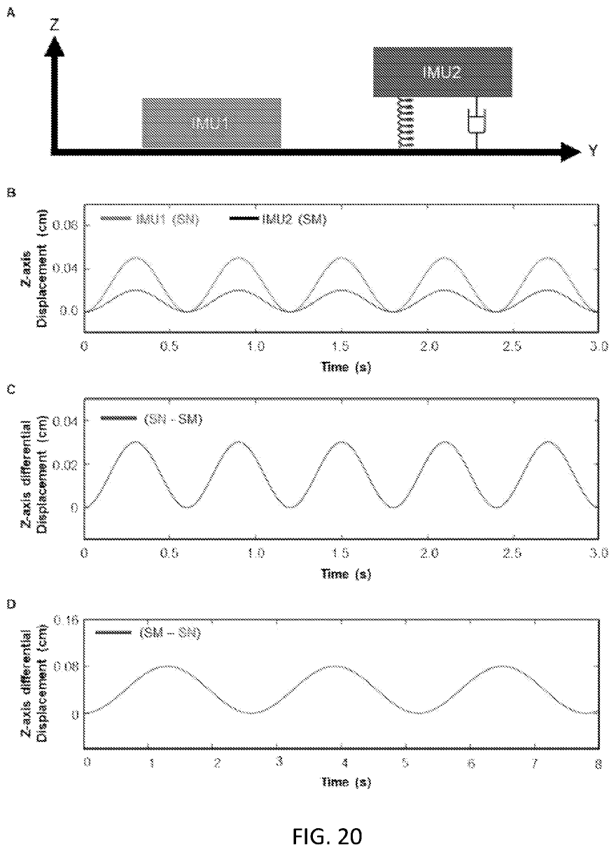

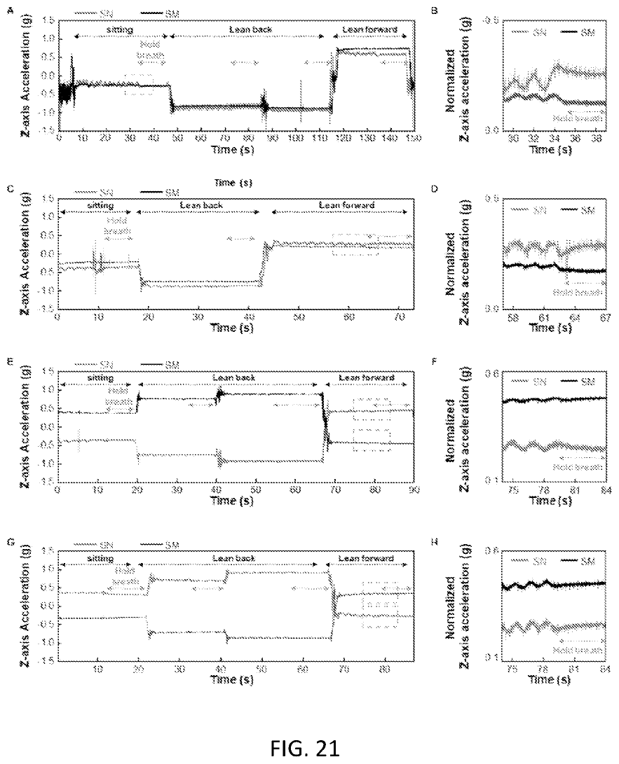

Similar considerations apply to differential dynamics associated with respiration. FIGS. 3F-3H and 18 (E-F) summarize the displacement distributions for three cycles of breathing while slightly swinging back and forth along the z axis. FIG. 3F shows motions at the SN and SM, where responses include contributions from body motions and respiration for each case. The differential result shown in FIG. 3G largely isolates the respiratory signals, as shown in FIG. 18 (E-F). Note that the small periodic features in these data arise from cardiac activity. A color contour plot of z-axis displacements at peak inhalation further highlights the spatial gradients that enable differential detection, as shown in FIG. 3H. Data captured with the devices show similar trends (FIG. 19), and simple 1D analytical models (FIG. 20; see the “Analytical modeling of differential accelerometry” section in Materials and Methods) can capture essential aspects of these behaviors (FIG. 21) that reveal a clear basis for differential detection at the SN and SM.

### Differential MA Sensing Minimizes Motion Artifacts in Respiratory and Cardiac Monitoring

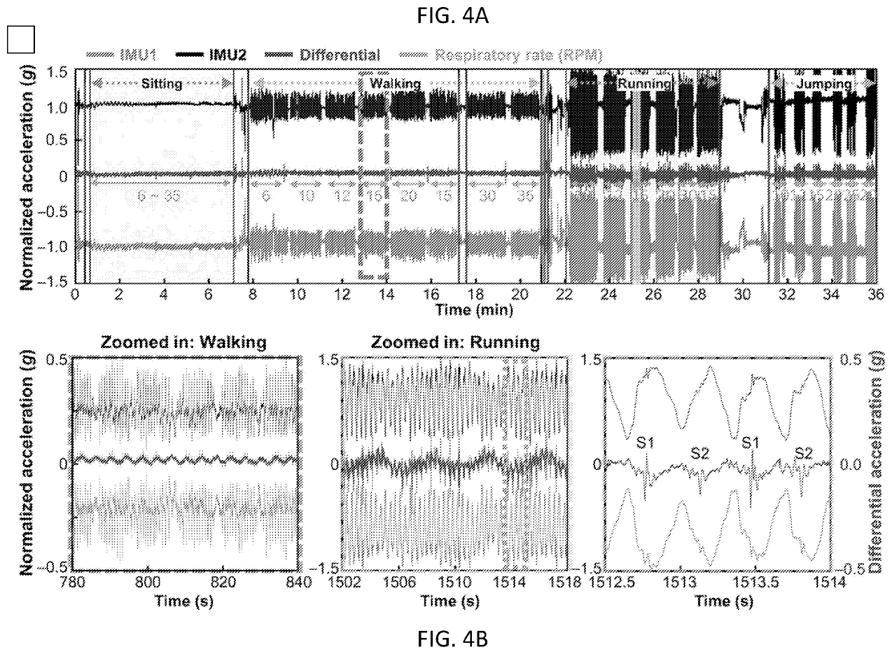

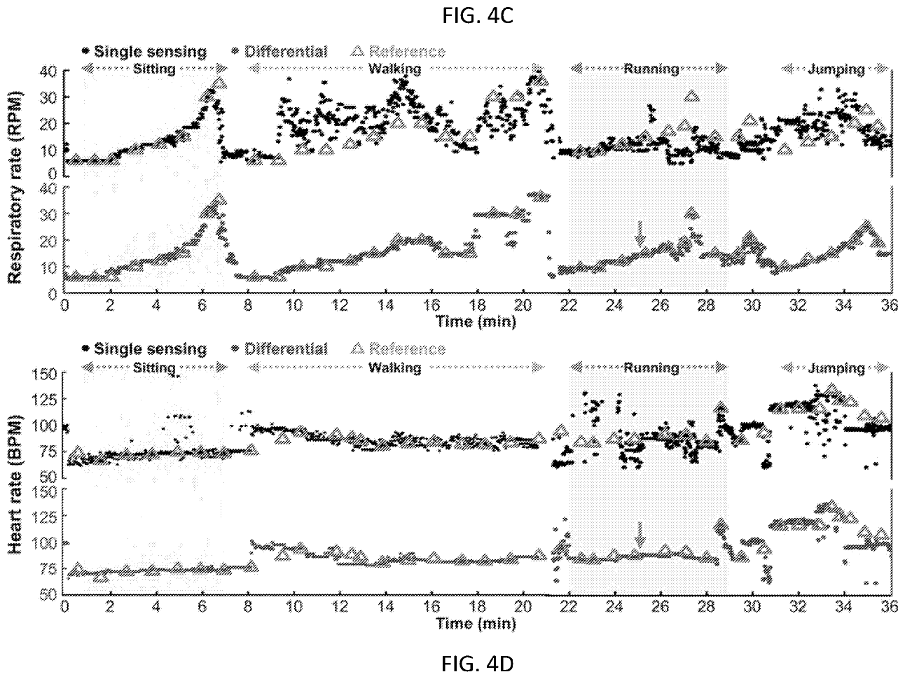

As verified by 3D-PTV, cardiac and respiratory activities create motions that have different amplitudes at the SN and the SM. Similar amplitudes result from movements of the core body. By consequence, simple subtraction of MA signals measured at these two locations greatly improves the accuracy and reliability of measurements of respiratory and cardiac activity by eliminating large, common-mode features that result from body motions. FIGS. 4A-4D summarizes results captured using a device platform that incorporates IMUs with capabilities in high-fidelity three-axial accelerometry.

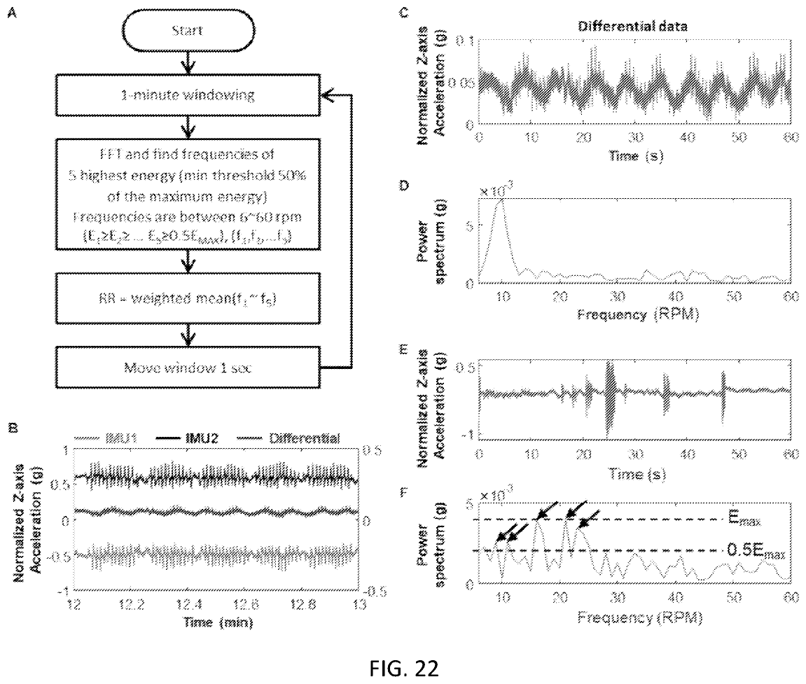

The flow chart in FIG. 22 (A) illustrates the approach for calculating the RR (respirations per minute (RPM)) from the differential data. The algorithm selects and performs a weighted average of the five highest energy components (minimum threshold of 50% of the maximum energy) in the frequency spectrum across the range of interest for the RR (6 to 60 RPM) within 1-min time windows, as shown in FIG. 22 (F). As shown in FIG. 22 (B-C), the differential signal largely eliminates common-mode “noise” associated with walking. Other signal components such as those due to cardiac activity lie outside the frequency range associated with respiration and/or have power below the threshold, as shown in FIG. 22 (D-E).

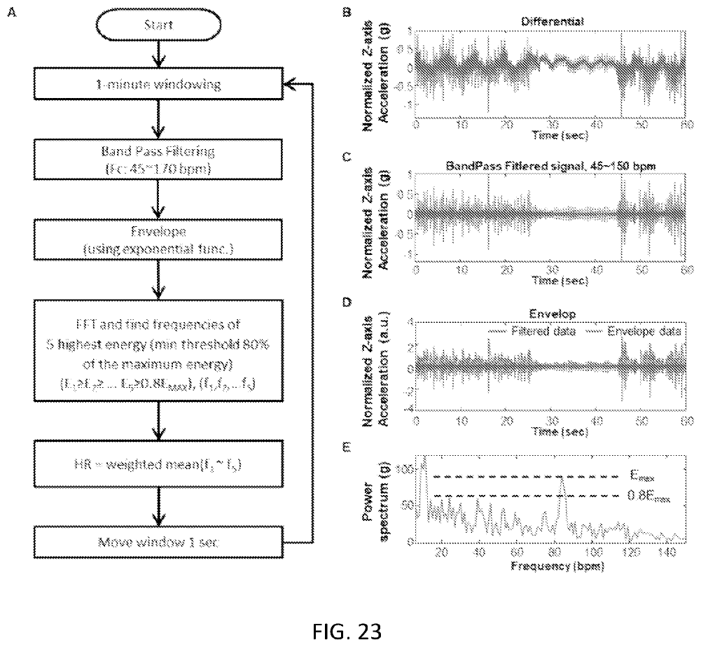

The flow chart in FIG. 23 (A) highlights the corresponding algorithm for HR (beats per minute (BPM)). As with the RR, the differential data remove features from walking, running, jumping, and related activities. Band-pass filtering of the frequency spectra for 1-min time windows with cutoff frequencies of 45 and 170 BPM eliminates low-frequency signals from slow body processes and high-frequency content from vocalization and related events, as shown in FIG. 23 (B-C). This frequency envelope captures essential features associated with the S1 peaks associated with cardiac sounds, equivalent to those observed in seismocardiograms (SCGs), as shown in FIG. 23 (D). The frequency with the maximum energy and those with at least 80% of the maximum energy serve (FIG. 23 (E)) as the basis for a weighted average to determine the HR.

FIGS. 4B-4D highlight results obtained during sitting, walking, running, and jumping. The first instance involves resting in a chair with a controlled RR of 6, 10, 12, 15, 20, 30, and 35 RPM (0 to 7 min). The subject intentionally controls the exhale/inhale (1:1 ratio) time with a timer while moving. Next, the subject walks (8 to 21 min, 90 steps/min with 50-cm average stride lengths), runs (22 to 29 min, 180 steps/min with 85-cm average stride lengths), and jumps (31 to 36 min, vertical jumps every 2 to 3 s at approximately 40-cm height), all under similar controlled RR. Walking and running generate repetitive sequences of high-amplitude, impulse signals that dominate the data from IMU1 (red) and IMU2 (black). The differential signals, by contrast, feature a clear, periodic response associated with respiration (15 BPM; blue), as shown in the left frame in FIG. 4B (purple dashed region in FIG. 4A) and the middle frame in FIG. 4B (yellow shaded region in FIG. 4A). This differential signal also contains information on cardiac activity, as prominent S1 and S2 peaks of an SCG (1.5 s; green dashed region in the middle frame in FIG. 4B).

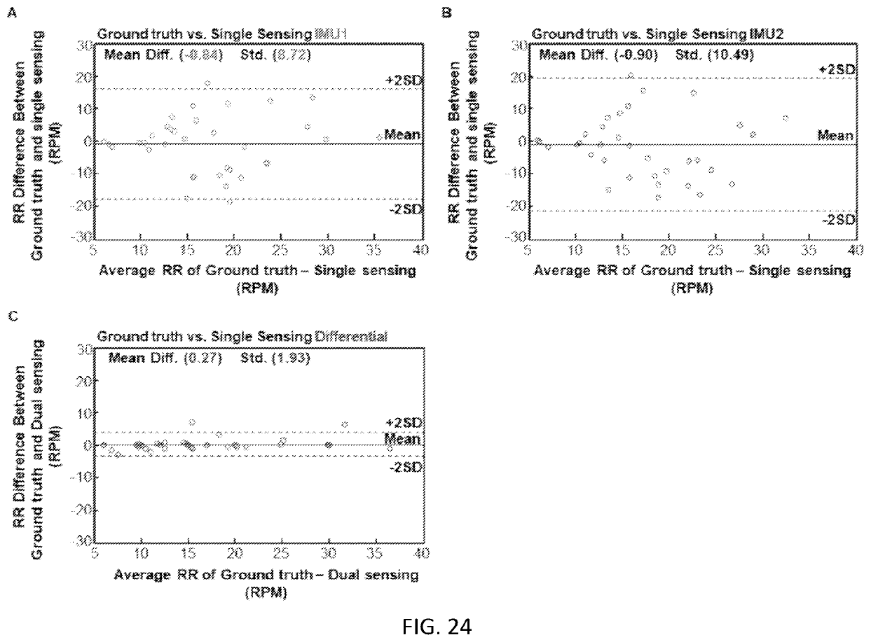

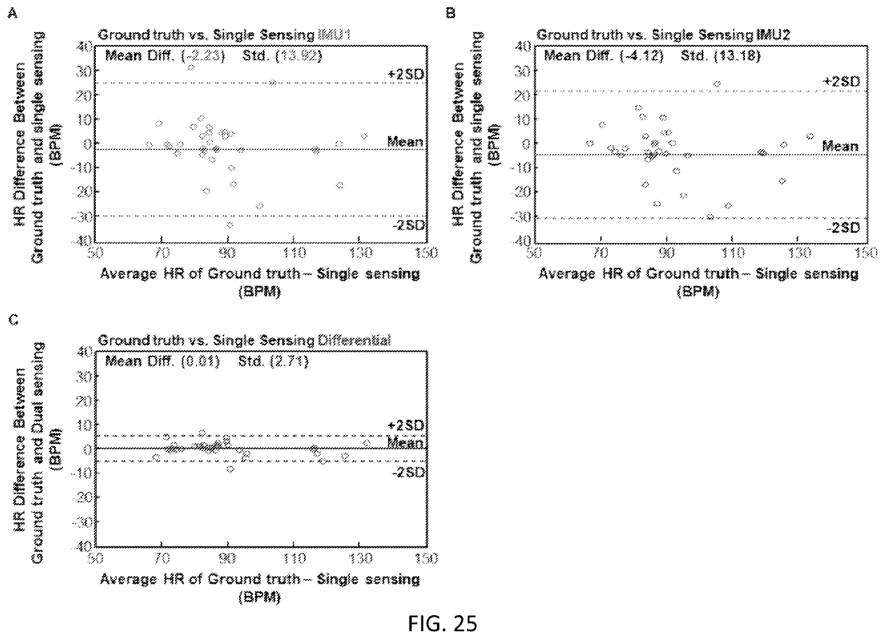

FIGS. 4C-4D compares RR and HR results extracted on the basis of normal and differential approaches. In the absence of body motions (e.g., sitting), the values are similar (blue shaded region in FIGS. 4C-4D). During ambulatory motions (walking, running, and jumping), results from single-accelerometer data (black dot) are highly variable compared to those from differential data (blue dot) for both HR and RR (yellow, red, and green shaded region in FIGS. 4C-4D. Differential sensing yields accurate results not only for walking and running but also for jumping. FIG. 24 (A-C) shows Bland-Altman plots for RR (single sensor, FIG. 24 (A-B); dual sensor, FIG. 24 (C)). The results with IMU1 (red) and IMU2 (black) exhibit a mean difference of −0.84 RPM (IMU1) and −0.90 RPM (IMU2) and an SD of 8.72 RPM (IMU1) and 10.49 RPM (IMU2). The differential results (blue) show a mean difference of 0.27 RPM and SD of 1.93 RPM. Likewise, for HR, results with IMU1 (red) and IMU2 (black) show a mean difference of −2.23 BPM (IMU1) and −4.12 BPM (IMU2) and an SD of 13.92 BPM (IMU1) and 13.18 (IMU2), and those with differential data (blue) show a mean difference of 0.01 BPM and an SD of 2.71 BPM (FIG. 25). When comparing single- and dual-sensing performance based on the SD of the extracted RR and HR, the differential signal from dual sensing shows an improvement of 77 and 79% over RR and HR from single-sensing data, respectively.

### Examples During Vigorous Activities in Sports

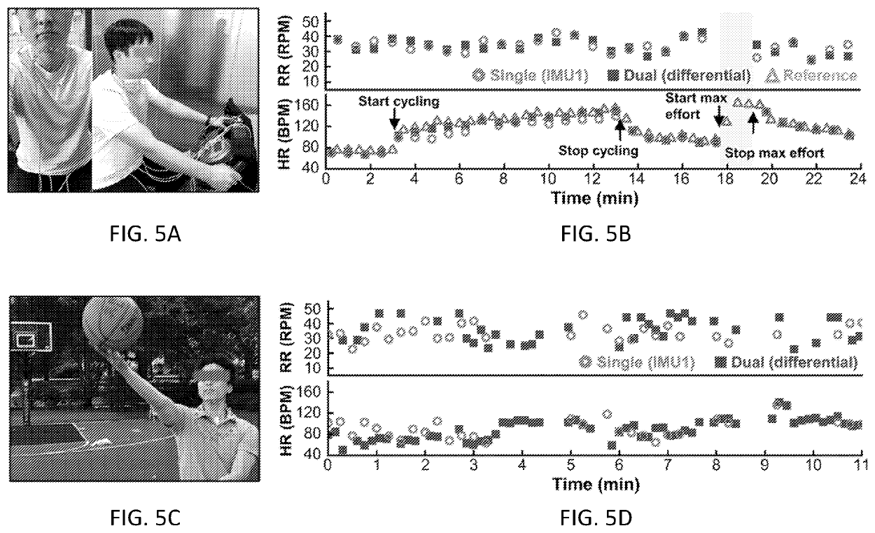

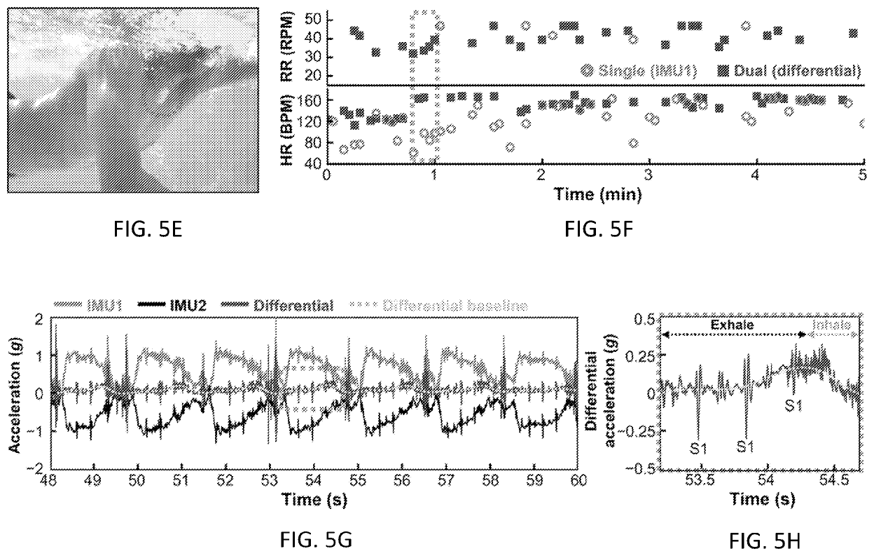

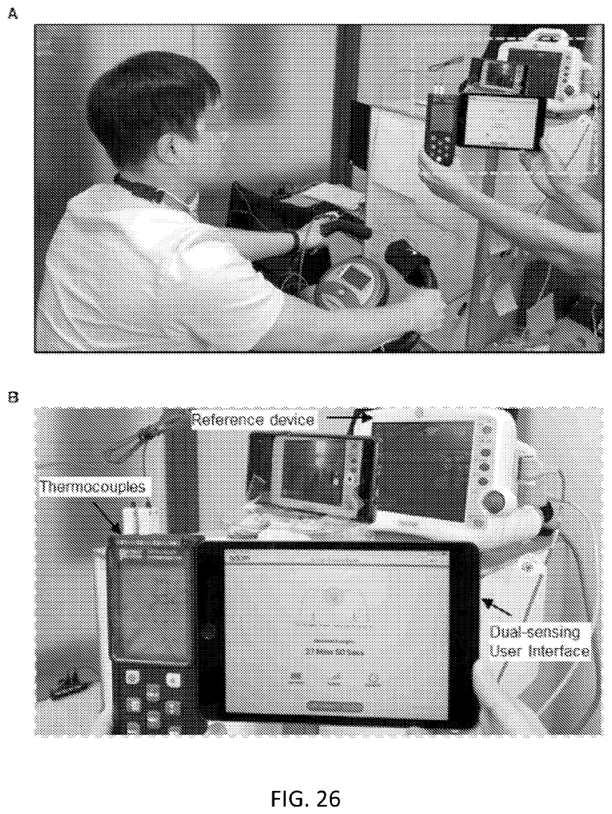

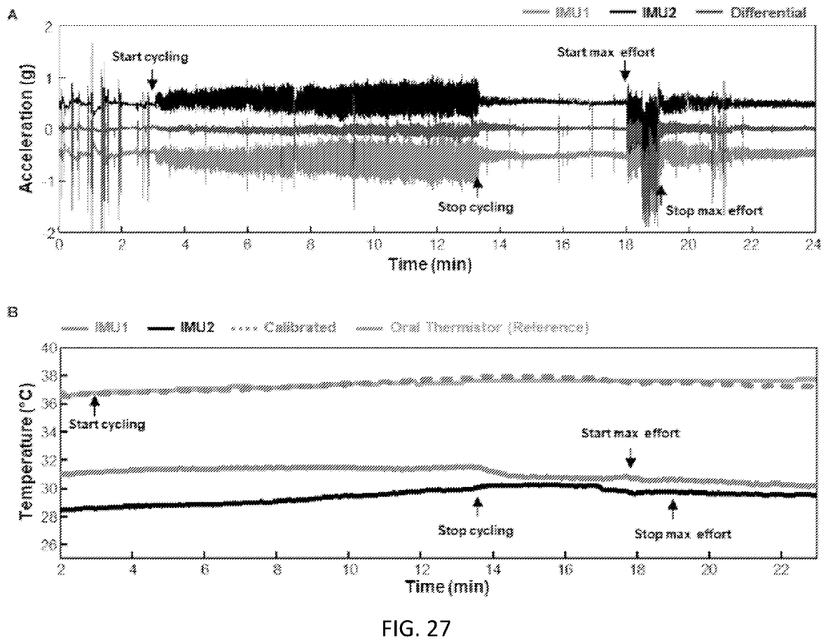

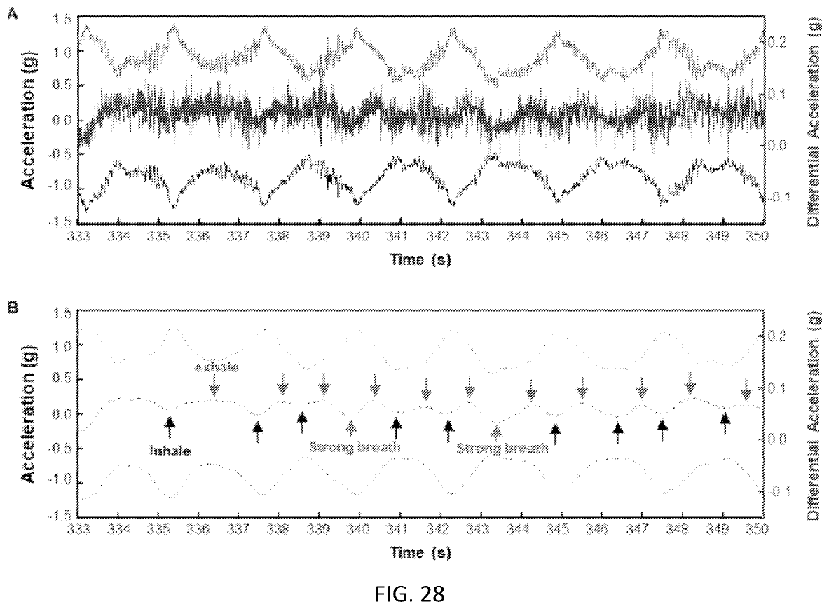

Athletic competition, fitness training, manual labor, and related activities create daunting challenges for accurate measurements of RR and HR because of fast, dynamic, and highly variable large-amplitude accelerations of the body. The dual-sensor platform offers powerful capabilities in these and other contexts. FIG. 5 highlights examples in cycling, playing basketball, and swimming. For exercise on a stationary bike (24 min; FIGS. 5A-5B, and 26-27), both the single-sensing (IMU1) and dual-sensing (differential) data yield HR results that match those obtained with a reference device (General Electronics, Dash3000). The RR values are also similar because of the limited effects of motion artifacts in this scenario. By contrast, for subjects playing basketball (11 min), differential sensing uniquely provides reliable measurements of HR and RR, as might be expected on the basis of controlled studies described previously (FIGS. 5C-Further benefits appear during extreme motions (3.5 to 5 min in FIG. 5D). The water-tight encapsulation and internal nonvolatile memory allows use in aquatic sports, as illustrated during swimming (5 min in FIG. 5E-5F). When motion artifacts and the target signal are in a similar frequency range, the differential measurement approach is particularly valuable. During swimming, RR calculations that use the signal from a single IMU are dominated by responses associated with swimming strokes. Our algorithm processes the results as outliers because of the large amplitudes of these accelerations, which are inconsistent with respiration, as shown in FIG. The differential signal from the dual sensor greatly minimizes signals associated with swimming strokes, thereby yielding clear features associated with cycles of exhalation and inhalation and enabling calculations of the RR. The differential signal yields accurate respiratory activity, although the patterns of breathing and swimming occur in the same frequency range, as shown in FIGS. 5G-5H. A similar demonstration, highlighted in FIG. 28, involves push-ups performed with controlled breathing out of phase with the push-up cycle, such that the RR signal cannot be distinguished from the periodic body motions using data from either IMU1 or IMU2. The differential signal, by contrast, shows clear features associated with exhalation/inspiration, well matched to the periodicity and the amplitude of controlled breathing.

Examples during Vigorous Activities in Manual Labor

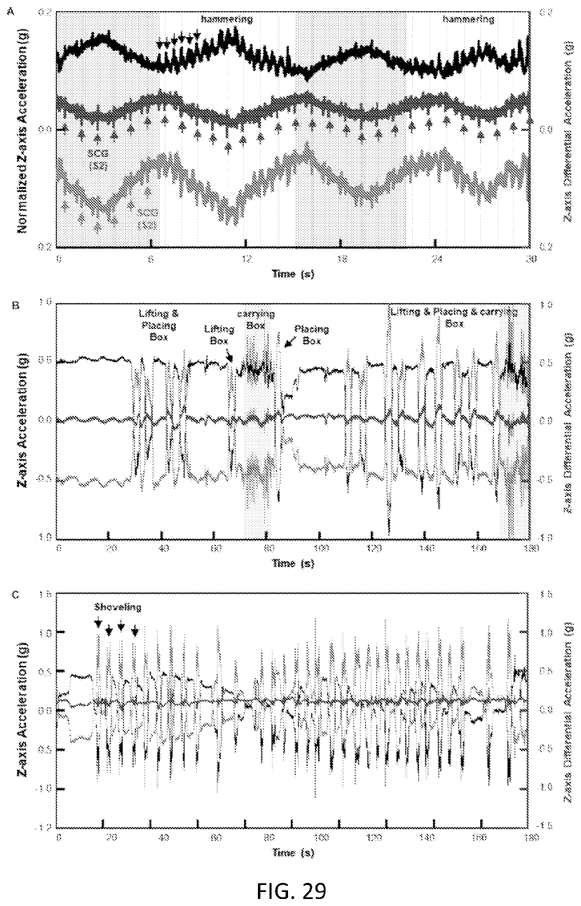

Worker health represents another area of opportunity given the need to continuously monitor key cardiopulmonary parameters in hostile environments. Demanding occupations that involve work in construction, mining, firefighting, and related areas could benefit from noninvasive, high-fidelity monitoring systems to detect fatigue, heat exhaustion, and performance in ways that are seamless and compatible with high motion artifacts and extreme ambient conditions in temperature, sounds, and other. FIG. 28 highlights examples of manual labor including hammering nails, carrying boxes, and shoveling dirt. Data in FIG. 29 (A) show that the differential signal exhibits clear features of cardiac activity, otherwise hidden by the strong, impulsive features associated with hammering. Similarly, respiratory features can be easily extracted even during large and irregular signatures of body movements in these cases, as shown in FIG. 29 (B-C).

### Clinical Deployment for Monitoring COVID-19 Patients Recover

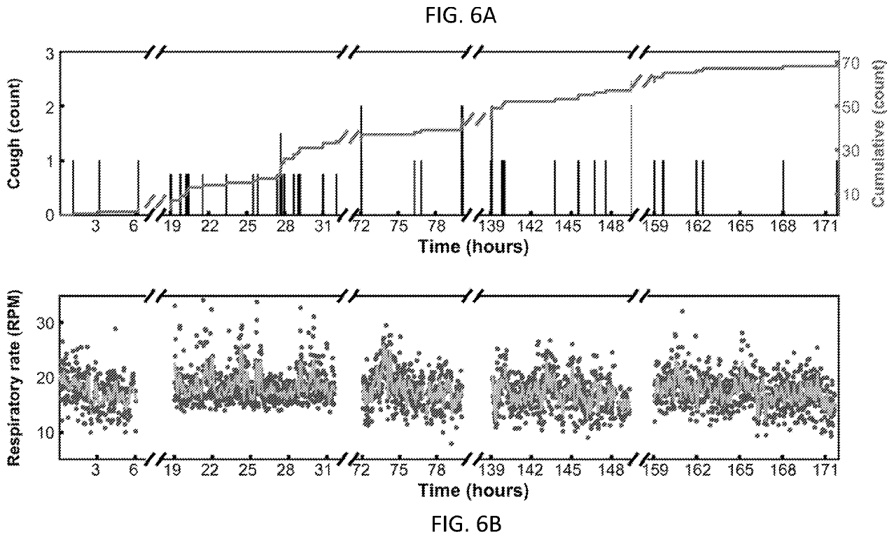

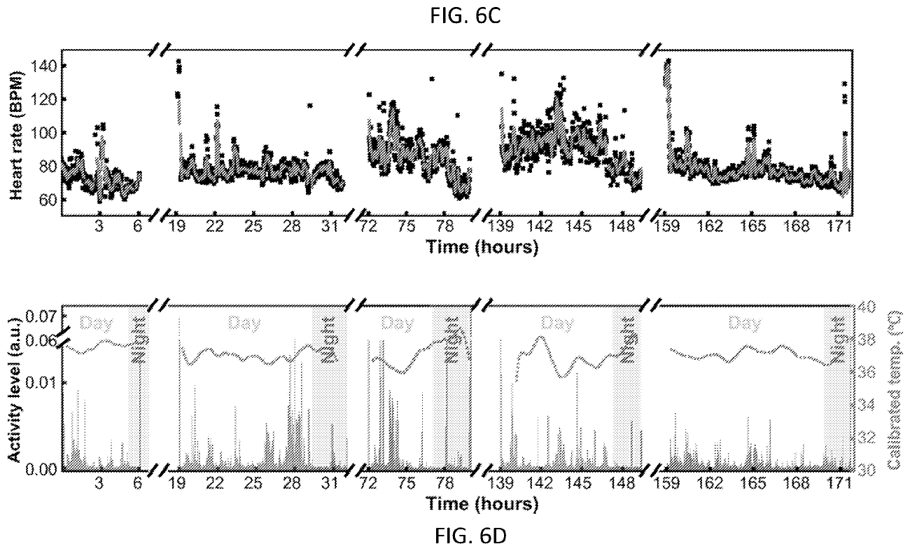

An area of urgent interest is in digital monitoring of the key symptoms of patients with COVID-19 to track the progress of recovery and the response to therapies in the hospital and the home. In addition to RR, HR, body movements, and body temperature, measurements also capture the intensity and frequency of coughing, talking, and laughing events. Collectively, these factors are important for symptomatic evaluation of the disease and for indirect assessments of aerosol production. Studies reported here involve a COVID-19-positive patient (49 years old; female; height, 170 cm; weight, 107 kg; type 2 diabetes mellitus, obesity, hypertension, and cerebrovascular accident in 2018) provided with the dual-sensing platform and instructions for use in recording over the course of 8 days, as shown in FIGS. 6A-6D. The patient captured 171 hours of data from 5 days within this time frame, including periods of dry cough and shortness of breath with oxygen therapy during the recovery phase. FIGS. 6A-6B show a decreasing trend in cumulative cough count, along with the RR during this same interval, where the blue dots represent 5-min averages and the orange line shows the data after processing with Savitzky-Golay smoothing. FIG. 6C summarizes the HR, where the black dots and red line show similar averages and smoothed results, respectively. FIG. 6D presents the activity level (green bar) calculated by integrating the spectral power across a frequency range from 1 to 10 Hz and the estimated core body temperature (red line). Daytime corresponds to the time interval between 6 a.m. and 6 p.m., while other times are considered night. Average body temperature recorded from the first day (37.5° C.) compared to the eighth recovery day (37.0° C.) shows a decrease of 0.5° C. This recovery period includes a regimented and intense set of physical rehabilitation protocols. The ability track vital signs and key symptoms throughout could provide actionable clinical information on recovery and patient readiness to return home. Widescale deployment of this technology could improve patient care, aid in managing the pandemic, and also enhance our understanding of the disease.

### Materials and Methods

### Encapsulating the Electronics in a Soft Enclosure

Top and bottom molded layers of a low-modulus elastomer (Silbione 4420; each 300 μm thick) form a soft encapsulating structure for the electronics. The fabrication process involved placing the electronics onto the bottom layer and then casting a uniform overcoat of a liquid prepolymer to a silicone elastomer (Ecoflex 0030). Mounting the top molded layer with a spacer on each of the short sides of the mold and clamping the assembly together enclosed the system for thermal curing at 70° C. in an oven for 20 min. Cooling to room temperature, removing the device, and eliminating excess elastomer from the perimeter using a die cutter completed the process.

### Forming the Thermal Insulating Foam

A three-axis milling machine (Roland MDX 540) created an aluminum mold with a concave shape. Casting a liquid precursor to a polyurethane foam material (mixing ratio of A to B is 2:3; FlexFoam-iT! III, Smooth-On, USA) on the mold after coating its surface with a releasing agent (Ease Release 200, Smooth-On, USA) and then pressing a flat aluminum plate on top side produced insulation foams upon curing on a hot plate at 100° C. for 30 min. A reflective film (thermal blanket; Swiss Safe Products) attached to the flat bottom surface of the foam layer using a 5-μm-thick double-sided tape (No. 5600, Nitto Denko Co., Japan) further improved the insulating properties. The final step of the process involved a CO2 laser (Universal Laser System Inc.) to cut the perimeter of the material into the final geometry.

### Modeling the Mechanical Characteristics

The commercial software ABAQUS (ABAQUS Analysis User's Manual 2010, version 6.10) defined the strain c in the metal layers of the system. The simulations allowed selection of design parameters to ensure that the strain in the copper (Cu) remains below the fracture limit (6=1%), to avoid mechanical failure during assembly of the device and during different types of deformations (stretching, bending, and twisting). The thin Cu and PI films were modeled by composite shell elements (S4R). The number of elements in the model was ˜2×10⁵, and the minimal element size was ⅛ of the width of the narrowest interconnects (100 μm). The mesh convergence of the simulation was guaranteed for all cases. The elastic modulus (E) and Poisson's ratio (v) of are E_{Cu}=119 GPa and v_{Cu}=0.34 for copper and E_{PI}=2.5 GPa and V_{PI}=0.34 for PI.

### 3D FEA Modeling for the Thermal Characteristics

Transient heat transfer analysis determined the effects of thermal conduction and natural convection on the responses of the temperature sensors. The tissue and internal sensor components were modeled by hexahedron elements (DC3D8). The encapsulation layer was modeled using tetrahedron elements (DC3D4). The number of elements in the model was ˜6×10⁵, and mesh convergence of the simulation was ensured for all cases. The boundary conditions included a constant temperature (T_{core}) at the bottom surface of the tissue layer and convection conditions with the ambient air (T_{amb}) at the free surfaces. The following parameters were used in the computations: room temperature T_{amb}=18° to 24° C.; convection coefficient h=(5 to 30) W m⁻² K⁻¹; thermal conductivity, heat capacity, and mass density of 0.3 W m⁻¹ K⁻¹, 1460 J kg⁻¹ K⁻¹, and 960 kg m⁻³ for the tissue; 0.21 W m⁻¹ K⁻¹, 1090 J kg′ K⁻¹, and 1420 kg m⁻³ for the thermoplastic chips; 0.343 W m⁻¹ K⁻¹, 1150 J kg⁻¹ K⁻¹, and 1850 kg m⁻³ for FR4; 0.03 W m⁻¹ K⁻¹, 1200 J kg′ K⁻¹, and 85 kg m⁻³ for the urethane foam; 0.21 W m⁻¹ K⁻¹, 2100 J kg′ K⁻¹, and 909 kg m⁻³ for PI; 0.2 W m⁻¹ K⁻¹, 1460 J kg′ K⁻¹, and 1070 kg m⁻³ for Ecoflex 00-30; and 0.15 W m⁻¹ K⁻¹, 1460 J kg′ K⁻¹, and 970 kg m′ for Silbione 4420.

### Measuring the Displacement Distributions by 3D-PTV

The experiments involved recordings from four synchronized high-speed area scan cameras (2048×1088 resolution; HT-2000M, Emergent) with 35-mm imaging lenses (F1.4 manual focus; Kowa) at the frame rate of 200 fps. The process focused on tracking of 300 fiducial points marked in a grid pattern across the neck covering the SN, the SM, and adjacent areas. The investigation volume was 10 cm by 8 cm by 10 cm illuminated by six arrays for 600 lumen light-emitting diode light bars. Preprocessing, calibration, 3D reconstruction, tracking, and postprocessing used customized 3D-PTV code. Image sequences were preprocessed by subtracting the background noise and enhancing the contrast. 3D calibration exploited the structure-from-motion technique from multiple views. After removing effects of lens distortion, intrinsic parameters of a single camera were estimated using the checkboard calibration method. Extrinsic parameters of all four cameras, including 3D translation and rotation matrices, were obtained by using a sparse set of points matched across the views. Once all camera parameters were estimated, a dense set of fiducial points across multiviews were detected in a subpixel level and reconstructed in 3D coordinate. 3D reconstructed fiducial points were tracked using the Hungarian algorithm and linked by performing a five-frame gap closing to produce long trajectories. Displacement, velocity, and Lagrangian acceleration were filtered and computed using fourth-order B splines. 3D displacement and vector contour fields were obtained by interpolating scattered fiducial points at each frame based on the Delaunay triangulation. Image sequences during cardiac activities were magnified using the Eulerian video magnification method.

### Procedures for Dual-Sensing Temperature and Motion Measurements

A double-sided medical silicone adhesive (3M, 2477P) secured the sensors to the neck area (aligned IMU1 on the SN and IMU2 on the SM). The authors affirm that all subjects in the study provided written informed consent for study images to be published with faces blurred. All data in this study were captured using IMUS with sensitivity of 0.061 mg (gravitational acceleration), 1666-Hz sampling rates (adjustable up to 6664 Hz), and ±2 g acceleration measurement range (adjustable up to ±16 g).

### Protocols for Human Subject Studies

The studies were approved by the Northwestern University Institutional Review Board, Chicago, IL, USA (STU00202449 and STU00212522) and were registered on ClinicalTrials.gov (NCT02865070 and NCT04393558). All study-related procedures were carried in accordance with the standards listed in the Declaration of Helsinki, 1964. For COVID-19-positive patients, double-sided medical silicone adhesive (3M, 2477P) secured the sensor to the neck area (aligned IMU1 on the SN and IMU2 on the SM) for more than 12 hours. For multiple days of use, medical-grade transparent film (Tegaderm, 3M) was applied between the skin and the double-sided adhesive to eliminate irritation from the adhesive. Clinical staff assisted the patient in placing the sensor. After each data measurement session, the device was sterilized with 70% isopropyl alcohol and left to dry at room temperature, and the sterilization process was repeated twice.

### Classifying Signal Features by Machine Learning to Extract Coughing Events

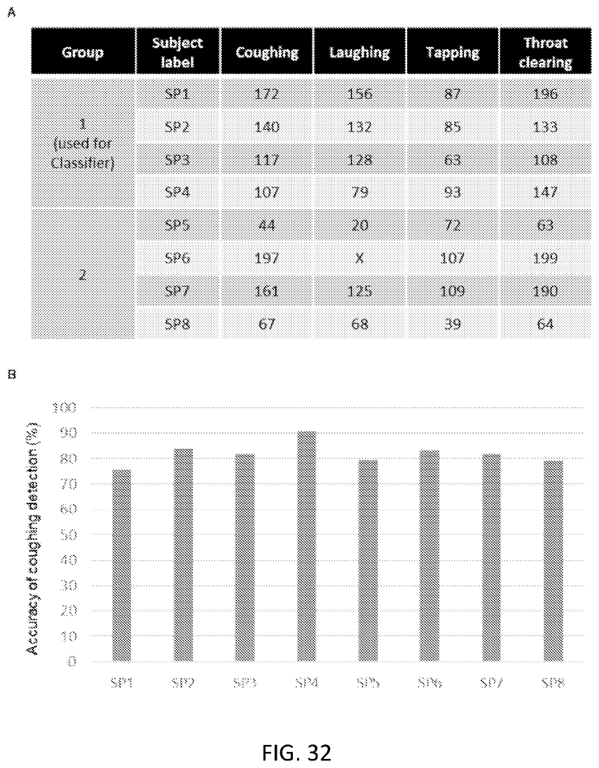

FIG. 31 shows a flow chart of the algorithm. Training data included time series z-axis acceleration data with features associated with tapping, coughing, laughing, and throat clearing. Training of this classifying algorithm used 10 datasets from each class (subjects SP1 to SP4), as shown in FIG. 32. Feature extraction used peak detection, spectral information, and spectrograms. The first step identified events associated with tapping, coughing, laughing, and throat clearing using adapted thresholds according to the input signal levels evaluated across sliding windows with widths of 0.5 s. Each extracted event was then aligned to the center of corresponding time frames to maximize the energy of the signal for postprocessing (first row of FIG. 31 (C)) based on continuous wavelet transformations. Resulting images within a 0.12-s window formed the basis for further analysis and classification (third row of FIG. 31 (C)). Specifically, a binary tree architecture using a support vector machine (SVM) classified these extracted features into four activities, as shown in FIG. 31 (D). First, in the SVM1 classifier, throat clearing activities were removed by negative values of the SVM1 hyperplane. Next, tapping activities were classified from SVM2 with a specific decision boundary (SVM2 result value: 2.5). Last, SVM3 separated the classes (coughing and laughing) with another decision boundary as described in FIG. 31 (E).

### 1-D Analytical Model for the Thermal Characteristics

It is assumed that the skin/tissue thickness and thermal properties are approximately the same for both sensors since the horizontal distance between them is relatively small. In this way, the core body temperature is determined from temperature variations through the thickness direction of the device. A 1-D heat transfer model based on the device material layers (thickness tᵢ and thermal conductivity kᵢ, listed in FIG. 14) was derived to estimate the core body temperature T_{core} from the temperature difference between the IMU sensors and the ambient temperature T_{amb}. The steady-state heat conduction equation to determine the temperature T in IMU1 and IMU2 is based on the equation

$$\frac{{\partial }^{2} T}{\partial {z}^{2}} = 0 ,$$

where z denotes the coordinate along the thickness direction in FIG. 14 (A).

The boundary conditions based on heat conduction through the device layers for the IMU1 sensor can be expressed as

$${{{{T}_{t} \vert}_{z = 0} = {T}_{c o r e} and {T}_{t} \vert}_{z = {t}_{t}} = {T}_{b s} \vert}_{z = {t}_{t}} \qquad ( 1 )$$

$${{[ {k}_{t} \frac{{dT}_{t}}{d z} ] \vert}_{z = {t}_{t}} = [ {k}_{s} \frac{{dt}_{b s}}{d z} ] \vert}_{z = {t}_{t}} \qquad ( 2 )$$

$${{{T}_{b s} \vert}_{z = {t}_{t} + {t}_{b s}} = {T}_{{IMU}_{1}} \vert}_{z = {t}_{t} + {t}_{b s}} , \qquad ( 3 )$$

$${{[ {k}_{s} \frac{d {T}_{b s}}{d z} ] \vert}_{z = {t}_{t} + {t}_{b s}} = [ {k}_{{IMU}_{1}} \frac{{dT}_{{IMU}_{1}}}{d z} ] \vert}_{z = {t}_{t} + {t}_{b s}}$$

$${{{T}_{{IMU}_{1}} \vert}_{z = {t}_{t} + {t}_{bs + {t}_{{IMU}_{1}}}} = {T}_{FR 4} \vert}_{z = {t}_{t} + {t}_{bs + {t}_{{IMU}_{1}}}} \qquad ( 4 )$$

$${{[ {k}_{{IMU}_{1}} \frac{d {T}_{{IMU}_{1}}}{d z} ] \vert}_{z = {t}_{t} + {t}_{b s + {t}_{{IMU}_{1}}}} = [ {k}_{FR 4} \frac{d {T}_{FR 4}}{d z} ] \vert}_{z = {t}_{y} + {t}_{b s + {t}_{{IMU}_{1}}}}$$

$${{{T}_{FR 4} \vert}_{z = {t}_{t} + {t}_{b s + {t}_{I M {U}_{1} + {t}_{FR 4}}}} = {T}_{f} \vert}_{z = {t}_{t} + {t}_{bs + {t}_{{IMU}_{1} + {t}_{FR 4}}}} \qquad ( 5 )$$

$${{[ {k}_{FR 4} \frac{{dT}_{PR 4}}{dz} ] \vert}_{z = {t}_{t} + {t}_{b s + {t}_{{IMU}_{1}} + {t}_{FR 4}}} = [ {k}_{f} \frac{{dT}_{f}}{dz} ] \vert}_{z = {t}_{y} + {t}_{b s + {t}_{{IMU}_{1}} + {t}_{PR 4}}}$$

$${{{T}_{f} \vert}_{z = {t}_{y} + {t}_{b s} + {t}_{I M {U}_{1}} + {t}_{F R 4} + {t}_{f}} = {T}_{t s} \vert}_{z = {t}_{t} + {t}_{b s} + {t}_{I M {U}_{1}} + {t}_{FR 4} + {t}_{f}} \qquad ( 6 )$$

$${{[ {k}_{f} \frac{{dT}_{f}}{d z} ] \vert}_{z = {t}_{t} + {t}_{bs} + {t}_{{IMU}_{1} + {t}_{FR 4} + {t}_{f}}} = [ {k}_{t s} \frac{{dT}_{ts}}{dz} ] \vert}_{z = {t}_{t} + {t}_{bs} + {t}_{{IMU}_{1} + {t}_{FR 4} + {t}_{f}}}$$

$${[ {k}_{s} \frac{{dT}_{t s}}{dz} ] \vert}_{z = {t}_{t} + {t}_{bs} + {t}_{{IMU}_{1} + {t}_{FR 4} + {t}_{f} + {t}_{s}}} = - h ( {T}_{ts} {\vert}_{z = {t}_{t} + {t}_{bs} + {t}_{{IMU}_{1} + {t}_{FR 4} + {t}_{f} + {t}_{s}}} - {T}_{amb} ) \qquad ( 7 )$$

Based on these boundary conditions the temperature of the IMU1 sensor can be expressed as

$${T}_{{IMU}_{1}} = {T}_{a m b} + \frac{{T}_{c o r e} - {T}_{amb}}{\frac{{t}_{t}}{{k}_{t}} + \frac{{t}_{b s}}{{k}_{s}} + \frac{{t}_{IMU 1}}{{k}_{IMU 1}} + \frac{{t}_{FR 4}}{{k}_{FR 4}} + \frac{{t}_{f}}{{k}_{f}} + \frac{{t}_{t s}}{{k}_{s}} + \frac{1}{h}} ( \frac{{t}_{t s}}{{k}_{s}} + \frac{{t}_{f}}{{k}_{f}} + \frac{{t}_{FR 4}}{{k}_{FR 4}} + \frac{1}{h} + \frac{{t}_{IMU 1}}{2 {k}_{IMU 1}} ) \qquad ( 8 )$$

Similarly, the boundary conditions based on the conduction through the device layers IMU2 sensor can be expressed as

$${{T}_{t} \vert}_{z = 0} = {T}_{core} \qquad ( 9 )$$

$${{{T}_{t} \vert}_{z = {t}_{t}} = {T}_{{t}_{bs}} \vert}_{z = {t}_{t}} , \qquad ( 10 )$$

$${{[ {k}_{t} \frac{d {T}_{t}}{d z} ] \vert}_{= {t}_{t}} = [ {k}_{s} \frac{{dT}_{b s}}{dz} ] \vert}_{z = {t}_{t}}$$

$${{{T}_{b s} \vert}_{z = {t}_{t} + {t}_{b s}} = {T}_{e} \vert}_{z = {t}_{t} + {t}_{b s}} \qquad ( 11 )$$

$${{[ {k}_{s} \frac{{dT}_{b s}}{dz} ] \vert}_{z = {t}_{t} + {t}_{b s}} = [ {k}_{e} \frac{d {T}_{e}}{d z} ] \vert}_{z = {t}_{t} + {t}_{b s}}$$

$${{{T}_{e} \vert}_{z = {t}_{t} + {t}_{bs + {t}_{e}}} = {T}_{t} \vert}_{z = {t}_{t} + {t}_{b s + {t}_{e}}} \qquad ( 12 )$$

$${{[ {k}_{e} \frac{{dT}_{e}}{dz} ] \vert}_{z = {t}_{t} + {t}_{b s + {t}_{e}}} = [ {k}_{f} \frac{{dT}_{f}}{dz} ] \vert}_{z = {t}_{t} + {t}_{b s + {t}_{e}}}$$

$${{{T}_{f} \vert}_{z = {t}_{t} + {t}_{b s + {t}_{e}} + {t}_{f}} = {T}_{{IMU}_{2}} \vert}_{z = {t}_{t} + {t}_{b s + {t}_{e}} + {t}_{f}} \qquad ( 13 )$$

$${{[ {k}_{f} \frac{{dT}_{f}}{dz} ] \vert}_{z = {t}_{t} + {t}_{b s + {t}_{e}} + {t}_{f}} = [ {k}_{{IMU}_{2}} \frac{{dT}_{{IMU}_{2}}}{dz} ] \vert}_{z = {t}_{t} + {t}_{b s + {t}_{e}} + {t}_{f}}$$

$${{{T}_{{IMU}_{2}} \vert}_{z = {t}_{t} + {t}_{b s + {t}_{e}} + {t}_{f} + {t}_{IMU 2}} = {T}_{t s} \vert}_{z = {t}_{t} + {t}_{b s + {t}_{e}} + {t}_{f} + {t}_{IMU 2}} \qquad ( 14 )$$

$${{[ {k}_{{IMU}_{2}} \frac{{dT}_{{IMU}_{2}}}{dz} ] \vert}_{z = {t}_{t} + {t}_{b s + {t}_{e}} + {t}_{f} + {t}_{IMU 2}} = [ {k}_{s} \frac{{dT}_{s}}{dz} ] \vert}_{z = {t}_{t} + {t}_{b s + {t}_{e}} + {t}_{f} + {t}_{IMU 2}}$$

$${[ {k}_{s} \frac{{dT}_{t s}}{dz} ] \vert}_{z = {t}_{t} + {t}_{b s + {t}_{e}} + {t}_{f} + {t}_{IMU 2} + {t}_{ts}} = - h ( {T}_{ts} {\vert}_{z = {t}_{t} + {t}_{b s + {t}_{e}} + {t}_{f} + {t}_{IMU 2} + {t}_{ts}} - {T}_{room} ) \qquad ( 15 )$$

$${T}_{{IMU}_{2}} = {T}_{a m b} + \frac{{T}_{core} - {T}_{amb}}{\frac{{t}_{t}}{{k}_{t}} + \frac{{t}_{bs}}{{k}_{s}} + \frac{{t}_{e}}{{k}_{e}} + \frac{{t}_{f}}{{k}_{f}} + \frac{{t}_{IMU 2}}{{k}_{IMU 2}} + \frac{{t}_{t s}}{{k}_{s}} + \frac{1}{h}} ( \frac{{t}_{t s}}{{k}_{s}} + \frac{1}{h} + \frac{{t}_{IMU 2}}{2 {k}_{IMU 2}} ) \qquad ( 16 )$$

FIG. 17 (A) shows that the temperature of IMU1 and IMU2 between 1-D analytical model and the 3-D FEA results agree well over the range of relevant heat convection coefficients h. The simplified 1-D model can be used to determine an expression for T_{core} by subtracting T_{IMU}_{₁}-T_{IMU}_{₂} as

$${T}_{Core} = {T}_{amb} + \frac{{T}_{{IMU}_{1}} - {T}_{{IMU}_{2}}}{( \frac{B}{A} - \frac{D}{C} )} \qquad ( 17 )$$

Where, the ratios (B/A) and (D/C) are given below and depend h.

$$\frac{B}{A} = \frac{\frac{{t}_{t s}}{{k}_{s}} + \frac{{t}_{t}}{{k}_{f}} + \frac{{t}_{FR 4}}{{k}_{FR 4}} + \frac{1}{h} + \frac{{t}_{IMU 1}}{2 {k}_{IMU 1}}}{\frac{{t}_{t}}{{k}_{t}} + \frac{{t}_{b s}}{{k}_{s}} + \frac{{t}_{IMU 1}}{{k}_{IMU 1}} + \frac{{t}_{F R 4}}{{k}_{F R 4}} + \frac{{t}_{f}}{{k}_{f}} + \frac{{t}_{ts}}{{k}_{s}} + \frac{1}{h}} \qquad ( 18 )$$

$$\frac{D}{C} = \frac{\frac{{t}_{t s}}{{k}_{s}} + \frac{1}{h} + \frac{{t}_{IMU 2}}{2 {k}_{IMU 2}}}{\frac{{t}_{t}}{{k}_{t}} + \frac{{t}_{b s}}{{k}_{s}} + \frac{{t}_{e}}{{k}_{e}} + \frac{{t}_{f}}{{k}_{f}} + \frac{{t}_{IMU 2}}{{k}_{IMU 2}} + \frac{{t}_{s}}{{k}_{s}} + \frac{1}{h}} \qquad ( 19 )$$

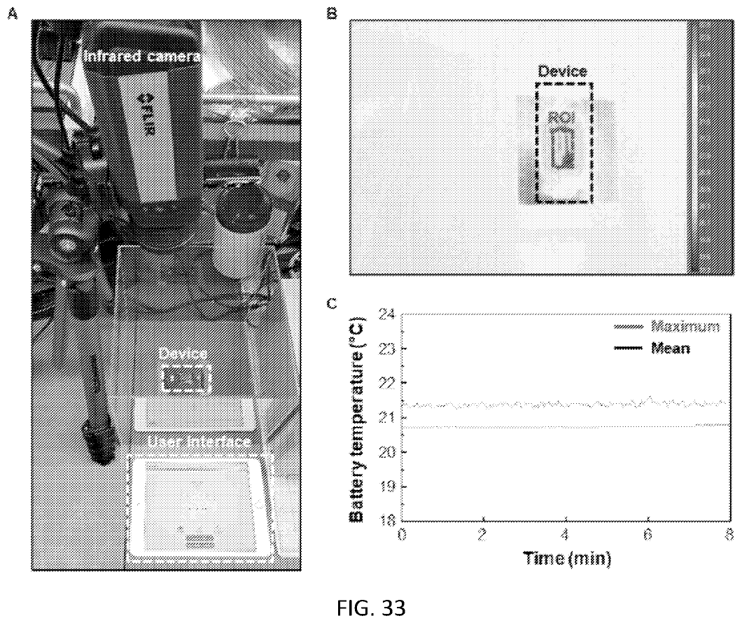

If the device is attached to a different body location with different thermal properties (e.g., neck, head, arm, etc.) then the skin/tissue thermal properties (i.e., thickness and thermal conductivity) would have to be adjusted accordingly (depending on the anatomy of the skin/tissue layers) for each location. Experiments show that the temperature of the battery changes by a negligible amount (<0.06° C.) during device operation (FIG. 33).

### Analytical Modeling of Differential Accelerometry

Although most of the capabilities in differential accelerometry arise from intrinsic differences in motions at the SN and SM, additional contributions can arise from details associated with the device layout. A schematic illustration of an analytical model that captures these structural differences is in FIG. 20. Here, all the components are treated as rigid bodies and only 1-D movement along the z-axis is considered. A harmonic displacement u applied to the rigid platform can be written as

u=A₀ sin(w₀t) (20)

to cause both sensors to accelerate along the z-axis. IMU1, located at the SN, is tied to the rigid platform (i.e. same displacement as equation (20)), considered as the chest wall. IMU2, at the SM, connects to this rigid platform by a spring of stiffness k with magnitude k=ΣᵢEᵢSᵢ/Lᵢ, where Eᵢ, Sᵢ, and Lᵢ are the Young modulus, effective area, and height, respectively, of different material/electronic layers between the platform and IMU2 (e.g. battery, device and silicone gel) and a damper with damping ratio ζ. Both IMU1 and IMU2 have a mass m. The damper in IMU2 provides a damping force proportional to the relative velocity F=−cv, where the damping ratio

$$ζ = \frac{c}{{c}_{c}} = \frac{c}{2 \sqrt{km}} ,$$

depends on the damping factor c and the critical damping factor c_{c}. In the dual sensor, silicone gel acts as a damper with a damping ratio ζ=1%˜12%. Since IMU1 is tied to the rigid platform, its acceleration is also the same as

a_{IMU1}=u̅=−w₀²A₀ sin(w₀t) (21)

The acceleration of IMU1 is described by the ordinary differential equation (ODE) kinematic equation

mx̅−=kΔx−cv=−k(x−A₀ sin(w₀t))−c(\dot{x}−\dot{u}) (22)

Equation (22) has a solution of the form

$$x = A {e}^{- w ζ t} sin ( {w}_{d} t + θ ) + b {A}_{0} sin ( {w}_{0} t - ε ) where w = \sqrt{\frac{k}{m}} , {w}_{d} = w \sqrt{1 - {ζ}^{2}} , \\ b = \sqrt{\frac{{w}^{4} + 4 {w}^{2} {w}_{0}^{2} {ζ}^{2}}{{( {w}^{2} - {w}_{0}^{2} )}^{2} + 4 {w}^{2} {w}_{0}^{2} {ζ}^{2}}} , \\ tan ( ε ) = \frac{2 {w}_{0}^{3} ζ}{{w}^{3} - {ww}_{0}^{2} + 4 {ww}_{0}^{2} {ζ}^{2}} . \qquad ( 23 )$$

The first term in equation (23) is the general solution and the second term is the solution. Using the initial conditions x(t=0)=0, \dot{x}(t=0)=0, the constants A and θ can be determined. For body motions, w₀=1˜3(2π) s⁻¹=6˜20 s⁻¹. For the “spring materials” (battery, gel, device) in the sensor, the total height is — 8 mm and the mass of accelerator is −0.07 g, sow should be larger than ˜1000 s⁻¹. The solution of A and θ is not relevant in this case since the first term will decay very fast in a few seconds, thereby simplifying equation (23) to

x=bA₀ sin(w₀t−ε) (24)

Therefore, the acceleration of IMU₂ can be determined as

a_{IMU2}=x̅=−bw₀²A₀ sin(w₀t−ε) (25)

The differential (IMU₁-IMU₂) is then

$${a}_{IMU 1} - {a}_{IMU 2} = - {w}_{0}^{2} {A}_{0} {b}_{0} sin ( {w}_{0} t - β ) where {b}_{0} = \sqrt{{b}^{2} - 2 b cos ( ε ) + 1} and tan ( β ) = \frac{b sin ( ε )}{1 - b cos ( ε )} \qquad ( 26 )$$

### Because

$$w ≫ {w}_{0} , tan ( ε ) = \frac{2 {w}_{0}^{3} ζ}{{w}^{3} - w {w}_{0}^{2} + 4 w {w}_{0}^{2} {ζ}^{2}} \to 0 ,$$

so ε→0 and there is no phase difference between IMU1 and IMU2. Meanwhile, b→1, so b₀→0 and the amplitude difference between IMU1 and IMU2 is very small.

### Discussion

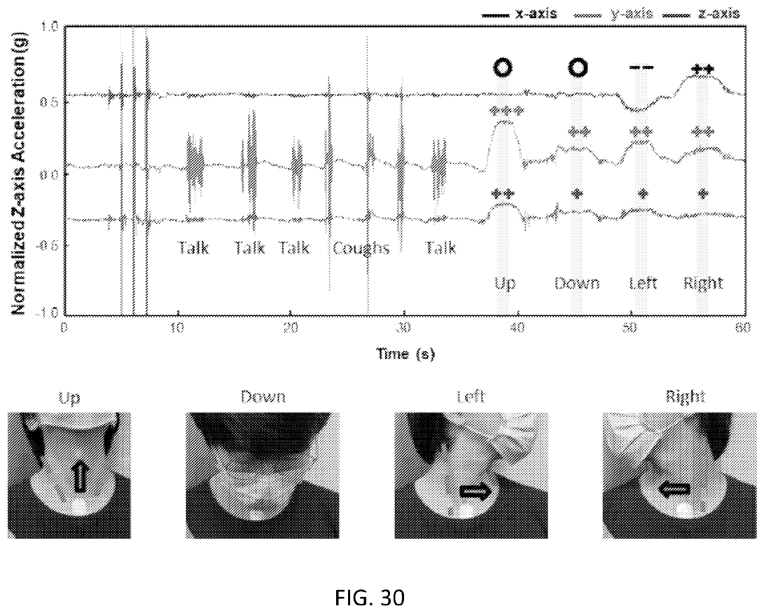

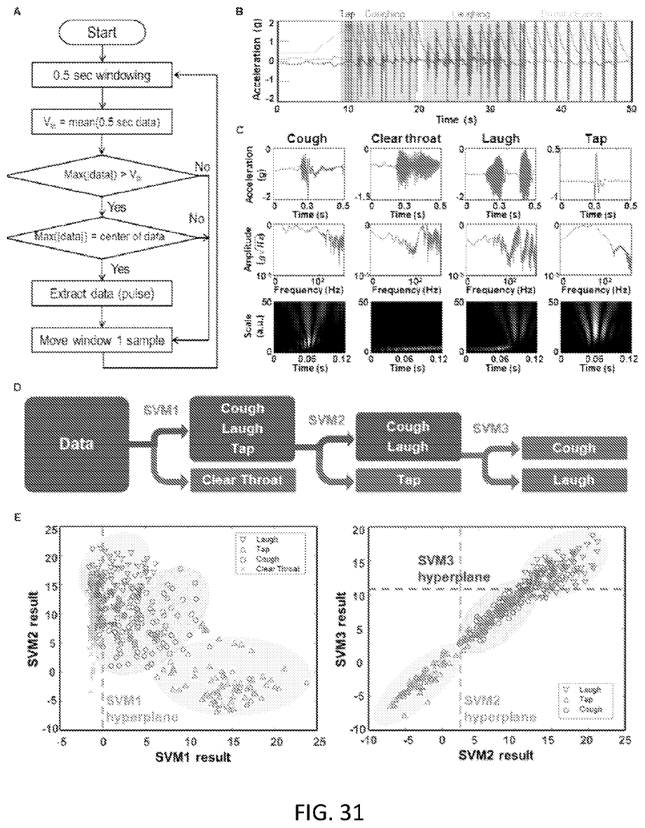

This exemplary example presents, among other things, a low-profile, lightweight, flexible, and wireless sensor that intimately couples to the skin as a dual measurement interface to the SN and SM with modalities for differential sensing of temperature and MA signatures of body processes. The results allow for measurements of a broad range of physiological parameters and activity behaviors that overcome a fundamental challenge in nearly every existing monitoring system: motion artifacts. Comparisons with previous studies on the mechano-acoustic sensing method are presented in Table 1. Specific examples reported here include tracking of cardiac activity, respiratory activity, respiratory sounds, body temperature, and overall activity across a range of controlled settings and natural activities in sports, manual labor, and clinical medicine. These technologies and underlying ideas have many other implications. Examples include rehabilitation for patients with aphasia and/or dysphagia, where measurements of vocal activity and swallowing are possible during daily life, outside hospitals or rehabilitation clinics, of particular relevance to stroke survivors and patients with chronic obstructive pulmonary disease. Capabilities in tracking these processes without privacy concerns associated with microphone recordings and in a manner that is independent of ambient sounds represent key features of the approach. The availability of multiaxial information, including three-axis acceleration measurements, three-axis gyroscope data, and three-axis magnetometer information, suggests additional opportunities for these same platforms. Examples include quantitative measurements of neck movements (FIG. 30) for patients recovering from cervical spine surgery by using accurate vector data between IMU1 and IMU2, as well as full-body motion detection followed by full-body motion reconstruction for rehabilitation or early-stage atypical motion diagnosis for cerebral palsy.

TABLE 1
Comparisons with previous studies on the mechano-acoustic
sensing method.

| | Studies | | |
|---|---|---|---|
| | Liu et al. | Lee et al. | This invention |
| Mechanics | Soft, | Soft, flexible | Soft, flexible |
| | flexible/Thin | | |
| Connectivity/ | Wired/ | Wireless BLE, | Wireless |
| Data | Local PC | Memory/ | BLE, Memory/ |
| management | | Local PC | Processing on Cloud |
| | | | platform for |
| | | | vital signals with |
| | | | machine learning |
| Sensors | Single IMU | Single IMU | Dual IMUs + Temp |
| | Amplifier | | |
| Features | Conformability | Placement | Motion noise |
| | on skin | (Suprasternal | canceling |
| | | notch) | Core body |
| | | | temp. estimation |
| | | | Placement |
| | | | (Suprasternal notch + |
| | | | sternal manubrium ) |
| Applications | Seismo- | Stagnant subject | Ambulatory |
| | cardiography | RR/HR | subject RR/HR |
| | Electro- | Swallow count | Time-sync with |
| | cardiography | Talking time | multiple devices |
| | Speech | Sleep study | Spatio-temporal |
| | recognition | | mapping (3D-PTV) |
| | | | Monitoring of |
| | | | vital signs |
| | | | during daily activities, |
| | | | vigorous exercises, |
| | | | intense manual labor |
| | | | COVID-19 |
| | | | patient recovery |
| | | | monitoring |
| Quantitative | | Only cycling | Walking + running + |
| comparison | | stationary | jumping (36 min) |
| | | bike (5 min) | HR: mean difference |
| | | HR: mean | of 0.01 BPM |
| | | difference | Standard deviation |
| | | of 3.1 BPM | of 2.71 BPM |
| | | Standard deviation | RR: mean difference |
| | | of 5.4 BPM | of 0.27 RPM |
| | | RR: mean difference | Standard deviation |
| | | of 0.3 RPM | of 1.93 RPM |
| | | Standard deviation | |
| | | of 2.5 RPM | |
| Ref. | [35] | [36] | This device |

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### REFERENCES

[1]. D.-H. Kim et al., Epidermal electronics. Science 333, 838-843 (2011).
[2]. S. Kabiri Ameri et al., Graphene electronic tattoo sensors. ACS Nano 11, 7634-7641 (2017).
[3]. A. Miyamoto, et al., Inflammation-free, gas-permeable, lightweight, stretchable on-skin electronics with nanomeshes. Nat. Nanotechnol. 12, 907-913 (2017).
[4]. J. Byun et al., Electronic skins for soft, compact, reversible assembly of wirelessly activated fully soft robots. Sci. Robot. 3, eaas9020 (2018).
[5]. S. Lee et al., Nanomesh pressure sensor for monitoring finger manipulation without sensory interference. Science 370, 966-970 (2020).
[6]. W.-H. Yeo et al., Multifunctional epidermal electronics printed directly onto the skin. Adv. Mater. 25, 2773-2778 (2013).
[7]. S. Xu et al., Soft microfluidic assemblies of sensors, circuits, and radios for the skin. Science 344, 70-74 (2014).
[8]. S. Choi et al., Highly conductive, stretchable and biocompatible Ag—Au core-sheath nanowire composite for wearable and implantable bioelectronics. Nat. Nanotechnol. 13, 1048-1056 (2018).
[9]. J. W. Ahn et al., A novel wearable EEG and ECG recording system for stress assessment. Sensors 19, 1991 (2019).
[10]. H. Jeong et al., Modular and reconfigurable wireless E-tattoos for personalized sensing. Adv. Mater. Technol. 4, 1900117 (2019).
[11]. H. U. Chung et al., Skin-interfaced biosensors for advanced wireless physiological monitoring in neonatal and pediatric intensive-care units. Nat. Med. 26, 418-429 (2020).
[12]. T. Yokota et al., Ultraflexible organic photonic skin. Sci. Adv. 2, e1501856 (2016).
[13]. T.-H. Kim et al., Fully stretchable optoelectronic sensors based on colloidal quantum dots for sensing photoplethysmographic signals. ACS Nano 11, 5992-6003 (2017).
[14]. H. Jeong et al., in 2017 39th Annual International Conference of the IEEE Engineering in Medicine and Biology Society (EMBC) (IEEE, 2017), pp. 4094-4097.
[15]. H. U. Chung et al., Binodal, wireless epidermal electronic systems with in-sensor analytics for neonatal intensive care. Science 363, eaau0780 (2019).
[16]. L. Y. Chen et al., Continuous wireless pressure monitoring and mapping with ultra-small passive sensors for health monitoring and critical care. Nat. Commun. 5, 5028 (2014).
[17]. N. Luo et al. et al., Flexible piezoresistive sensor patch enabling ultralow power cuffless blood pressure measurement. Adv. Funct. Mater. 26, 1178-1187 (2016).
[18]. J. Kim et al., Soft wearable pressure sensors for beat-to-beat blood pressure monitoring. Adv. Healthc. Mater. 8, 1900109 (2019).
[19]. M. Kaisti et al., Clinical assessment of a non-invasive wearable MEMS pressure sensor array for monitoring of arterial pulse waveform, heart rate and detection of atrial fibrillation. NPJ Digit. Med. 2, 39 (2019).
[20]. H. Yao et al., Environment-resilient graphene vibrotactile sensitive sensors for machine intelligence. ACS Mater. Lett. 2, 986-992 (2020).
[21]. D. J. Lipomi et al., Skin-like pressure and strain sensors based on transparent elastic films of carbon nanotubes. Nat. Nanotechnol. 6, 788-792 (2011).
[22]. C.-S. Kim et al., Ballistocardiogram as proximal timing reference for pulse transit time measurement: Potential for cuffless blood pressure monitoring. IEEE Trans. Biomed. Eng. 62, 2657-2664 (2015).
[23]. W. Gao et al., Fully integrated wearable sensor arrays for multiplexed in situ perspiration analysis. Nature 529, 509-514 (2016).
[24]. H. Lee et al., A graphene-based electrochemical device with thermoresponsive microneedles for diabetes monitoring and therapy. Nat. Nanotechnol. 11, 566-572 (2016).
[25]. Y. Yamamoto et al., Printed multifunctional flexible device with an integrated motion sensor for health care monitoring. Sci. Adv. 2, e1601473 (2016).
[26]. M. Bariya et al., Wearable sweat sensors. Nat. Electronics 1, 160-171 (2018).
[27]. M. Elsherif et al., Wearable contact lens biosensors for continuous glucose monitoring using smartphones. ACS Nano 12, 5452-5462 (2018).
[28]. J. Kim et al., Simultaneous monitoring of sweat and interstitial fluid using a single wearable biosensor platform. Adv. Sci. 5, 1800880 (2018).
[29]. S. K. Ameri et al., Imperceptible electrooculography graphene sensor system for human-robot interface. NPJ 2D Mater. Appl. 2, 19 (2018).
[30]. L. Dejace et al., Gallium-based thin films for wearable human motion sensors. Adv. Intell. Syst. 1, 1900079 (2019).
[31]. L. Tian et al., Large-area MRI-compatible epidermal electronic interfaces for prosthetic control and cognitive monitoring. Nat. Biomed. Eng. 3, 194-205 (2019).
[32]. M. Chu et al., Respiration rate and volume measurements using wearable strain sensors. NPJ Digit. Med. 2, 8 (2019).
[33]. X.-R. Ding et al., Wearable sensing and telehealth technology with potential applications in the coronavirus pandemic. IEEE Rev. Biomed. Eng. 14, 48-70 (2020).
[34]. S. Kim et al., Soft, skin-interfaced microfluidic systems with integrated immunoassays, fluorometric sensors, and impedance measurement capabilities. Proc. Natl. Acad. Sci. U.S.A. 117, 27906-27915 (2020).
[35]. Y. Liu, et al., Epidermal mechano-acoustic sensing electronics for cardiovascular diagnostics and human-machine interfaces. Sci. Adv. 2, e1601185 (2016).
[36]. K. Lee et al., Mechano-acoustic sensing of physiological processes and body motions via a soft wireless device placed at the suprasternal notch. Nat. Biomed. Eng. 4, 148-158 (2020).
[37]. H. Jeong et al., Continuous on-body sensing for the COVID-19 pandemic: Gaps and opportunities. Sci. Adv. 6, eabd4794 (2020).
[38]. M. J. Mathie et al., Accelerometry: Providing an integrated, practical method for long-term, ambulatory monitoring of human movement. Physiol. Meas. 25, R1-R20 (2004).
[39]. F. Leitäo et al., High-resolution seismocardiogram acquisition and analysis system. Sensors 18, 3441 (2018).
[40]. T. Ha et al., A chest-laminated ultrathin and stretchable e-tattoo for the measurement of electrocardiogram, seismocardiogram, and cardiac time intervals. Adv. Sci. 6, 1900290 (2019).
[41]. A. Dragomir et al., Acoustic detection of coronary occlusions before and after stent placement using an electronic stethoscope. Entropy 18, 281 (2016).
[42]. Y. Huang et al., Real-time intended knee joint motion prediction by deep-recurrent neural networks. IEEE Sensors J. 19, 11503-11509 (2019).
[43]. C. Adans-Dester et al., Enabling precision rehabilitation interventions using wearable sensors and machine learning to track motor recovery. NPJ Digit. Med. 3, 121 (2020).
[44]. CDC, Communities, Schools, Workplaces, &amp; Events. Centers for Disease Control and Prevention (2020); www.cdc.gov/coronavirus/2019-ncov/community/clean-disinfect/index.html.
[45]. O. Kimberger et al., Accuracy and precision of a novel non-invasive core thermometer. Br. J. Anaesth. 103, 226-231 (2009).
[46]. O. Kimberger et al., The accuracy of a disposable noninvasive core thermometer. Can. J. Anesth. 60, 1190-1196 (2013).
[47]. Y. Zhang et al., Theoretical and experimental studies of epidermal heat flux sensors for measurements of core body temperature. Adv. Healthc. Mater. 5, 119-127 (2016).
[48]. J.-T. Kim, L. P. Chamorro, Lagrangian description of the unsteady flow induced by a single pulse of a jellyfish. Phys. Rev. Fluids 4, 064605 (2019).
[49]. J.-T. Kim et al., On the dynamics of air bubbles in Rayleigh-Benard convection. J. Fluid Mech. 891, A7 (2020).
[50]. I. Awolusi et al., Wearable technology for personalized construction safety monitoring and trending: Review of applicable devices. Autom. Constr. 85, 96-106 (2018).
[51]. E. J. Chow et al., Symptom screening at illness onset of health care personnel with SARS-CoV-2 infection in King County, Washington. JAMA 323, 2087-2089 (2020).
[52]. K. Y. Chen, D. R. Bassett, The technology of accelerometry-based activity monitors: Current and future. Med. Sci. Sports Exerc. 37, S490-S500 (2005).
[53]. S. Brage et al. et al., Estimation of free-living energy expenditure by heart rate and movement sensing: A doubly-labelled water study. PLOS ONE 10, e0137206 (2015).
[54]. H.-Y. Wu et al., Eulerian video magnification for revealing subtle changes in the world. ACM Trans. Graph. 31, 1-8 (2012).
[55]. P. S. L. Anderson et al., Taking a stab at quantifying the energetics of biological puncture. Integr. Comp. Biol. 59, 1586-1596 (2019).
[56]. J.-T. Kim et al., Free fall of homogeneous and heterogeneous cones. Phys. Rev. Fluids 093801 (2020).

1. An electronic device for measuring physiological parameters of a living subject, comprising:
at least a first inertial measurement unit (IMU) and a second IMU, the first IMU and the second IMU are time-synchronized to and spatially and mechanically separated from each other; and
a microcontroller unit (MCU) electronically coupled to the first IMU and the second IMU for processing of data streams from the first IMU and the second IMU.

2. The electronic device of claim 1, wherein the first IMU is configured to measure data including a first signal related to a physiological signal of the living subject and a second signal, and the second IMU is configured to measure data including at least the second signal, wherein the first signal measured by the first IMU has a signal strength greater than that the second signal measured by the first IMU.

3. The electronic device of claim 2, wherein the data measured by the first IMU and the second IMU are processed such that subtraction of the second signal measured by the second sensor from the second signal measured by the first sensor results in a stronger first signal that is a signal of interest.

4. The electronic device of claim 2, wherein the second signal is related to at least one of ambient, motion and vibration.

5. The electronic device of claim 2, wherein the data measured by the second IMU includes the first signal and the second signal.

6. The electronic device of claim 2, wherein a signal-to-noise ratio (SNR) of a signal measured by the first IMU and the second IMU together is lower than a first SNR of a signal measured by the first IMU individually, or a second SNR of a signal measured by the second IMU individually.

7. The electronic device of claim 2, wherein both of the first IMU and the second IMU are operably in mechanical communication with the skin of the living subject.

8. The electronic device of claim 7, wherein one of the first IMU and the second IMU is operably in directly mechanical communication with the skin of the living subject, while the other of the first IMU and the second IMU is operably in indirectly mechanical communication with the skin of the living subject.

9. The electronic device of claim 8, wherein the first IMU and the second IMU are operably in directly mechanical communication with the skin of the living subject.

10. The electronic device of claim 2, wherein one of the first IMU and the second IMU is separated from the rest of rigid components of the electronic device.

11. The electronic device of claim 1, further comprising at least a first thermal sensing unit and a second thermal sensing unit, wherein one of the first and second thermal sensing units is thermally isolated from an ambient environment and configured to measure a body temperature of the living subject, and the other of the first and second thermal sensing units is configured to measure the ambient temperature.

12. The electronic device of claim 11, wherein each of the first and second thermal sensing units is embedded in a respective one of the first and second IMUs.

13. The electronic device of claim 1, being configured to measure a range of physiological information from activity of a cardiopulmonary system and movements of a core body to a diverse collection of processes across thoracic cavity, esophagus, pharynx, and oral cavity related to respiration, speech, swallowing, wheezing, coughing, and sneezing.

14. The electronic device of claim 13, being configured to separate signals associated with the cardiopulmonary system and related processes from those due to body movements.

15. The electronic device of claim 13, being configured to spatiotemporally map movements of the skin at this region of the anatomy onto which the electronic device is attached during cardiac and respiratory activities.

16. The electronic device of claim 13, being configured to continuously measure temperature, heart rate (HR), respiratory rate (RR), activity level, and body orientation, across a range of vigorous activities and conditions.

17. The electronic device of claim 13, being configured to monitor key symptoms of a patient with COVID-19 infection to track progress of recovery and response to therapies in hospital and/or home.

18. The electronic device of claim 13, being configured to measure any of respiratory or motion related digital biomarkers associated with coughing, swallowing, and/or specific motion related activities.

19. The electronic device of claim 18, being configured to assess coughing when the living subject is moving or immobile, and/or to measure muscle motion, when the living subject is moving.

20. The electronic device of claim 1, further comprising a bidirectional wireless communication system electronically coupled to the electronic device and configured to send an output signal from the electronic device to an external device.

21. The electronic device of claim 20, wherein the bidirectional wireless communication system is further configured to deliver commands from the external device to the electronic device.

22. The electronic device of claim 20, wherein the bidirectional wireless communication system comprises a controller that utilizes at least one of near field communication (NFC), Wi-Fi/Internet, Bluetooth, Bluetooth low energy (BLE), and cellular communication protocols for wireless communication.

23. The electronic device of claim 20, further comprising a customized app with a user interface deployed in the external device to allow a user to configure and operate the electronic device for data collection, data transfer, data storage and analysis, wireless charging, and monitoring of user's conditions.

24. The electronic device of claim 23, wherein the customized app is configured to allow time-synchronized operation of a plurality of the electronic devices simultaneously.

25. The electronic device of claim 20, wherein the external device is a mobile device, a computer, or a cloud service.

26. The electronic device of claim 1, further comprising a power module coupled to the first IMU, the second IMU and the MCU for providing power thereto.

27. The electronic device of claim 26, wherein the power module further comprises a failure prevention element including a short-circuit protection component or a circuit to avoid battery malfunction.

28. The electronic device of claim 26, wherein the power module comprises at least one battery for providing the power.

29. The electronic device of claim 28, wherein the battery is a rechargeable battery.

30. The electronic device of claim 29, wherein the power module further comprises a wireless charging module for wirelessly charging the rechargeable battery.

31. The electronic device of claim 28, wherein the second IMU is placed in a manner that it bends and folds over the battery.

32. The electronic device of claim 26, further comprising a flexible printed circuit board (fPCB) having flexible and stretchable interconnects electrically connecting to electronic components including the first IMU, the second IMU and the MCU and the power module.

33. The electronic device of claim 32, further comprising an elastomeric encapsulation layer at least partially surrounding the electronic components and the flexible and stretchable interconnects to form a tissue-facing surface operably attached to the living subject and an environment-facing surface, wherein the tissue-facing surface is configured to conform to a skin surface of the living subject.

34. The electronic device of claim 33, wherein the encapsulation layer is formed of a flame retardant material.

35. The electronic device of claim 34, wherein the elastomeric encapsulation layer is a waterproof and biocompatible silicone enclosure.

36. The electronic device of claim 1, further comprising a biocompatible hydrogel adhesive for attaching the electronic device on the respective region of the living subject, wherein the biocompatible hydrogel adhesive is adapted such that signals from the living subject are operably conductible to the first IMU and the second IMU.

37. The electronic device of claim 1, being flexible and conformable to the skin with a specific geometrical polarity for mounting in an anatomical location of interest of the living subject.

38. The electronic device of claim 1, being a wearable, twistable stretchable, and/or bendable.

39. An electronic device for measuring physiological parameters of a living subject, comprising:
a sensor network comprising a plurality of sensor units operably deployed on a skin of the living subject, the plurality of sensor units being time-synchronized to and spatially and mechanically separated from each other; and
a microcontroller unit (MCU) electronically coupled to the plurality of sensor units for processing of data streams from the plurality of sensor units.

40. The electronic device of claim 39, wherein the plurality of sensor units are configured to measure a same physiological parameter, or different physiological parameters.

41. The electronic device of claim 39, wherein each of the plurality of sensor units comprises at least a first sensor and a second sensor time-synchronized to and spatially and mechanically separated from each other.

42. The electronic device of claim 41, wherein for each sensor unit, the first sensor is configured to measure data including a first signal related to a physiological signal of the living subject and a second signal, and the second sensor is configured to measure data including at least the second signal, wherein the first signal measured by the first sensor has a signal strength greater than that the second signal measured by the first sensor.

43. The electronic device of claim 42, wherein the data measured by the first sensor and the second sensor of said sensor unit are processed such that subtraction of the second signal measured by the second sensor from the second signal measured by the first sensor results in a stronger first signal that is a signal of interest.

44. The electronic device of claim 42, wherein the second signal is related to at least one of ambient, motion and vibration.

45. The electronic device of claim 41, wherein each of the first sensor and the second sensor comprises an inertial measurement unit (IMU), a thermal sensor, a pressure sensor, and/or optical sensor.

46. The electronic device of claim 45, wherein each of the first sensor and the second sensor comprises the IMU.

47. The electronic device of claim 46, wherein further comprising a plurality of thermal sensing units.

48. The electronic device of claim 47, wherein each thermal sensing units is embedded in a respective IMU.

49. The electronic device of claim 47, wherein the MCU operably receives synchronized outputs of the plurality of thermal sensor units with at least one thermal sensing unit for an ambient environment and at least one thermal sensing unit in direct thermal communication from the body isolated thermally from the ambient environment with in-sensor thermally isolating materials.

50. The electronic device of claim 39, being configured to automatically switch operation modes, wherein the operation modes include at least a first mode when the living subject is at rest, and a second modes when the living subject is in a high motion.

51. The electronic device of claim 39, being configured to measure a range of physiological information from activity of a cardiopulmonary system and movements of a core body to a diverse collection of processes across thoracic cavity, esophagus, pharynx, and oral cavity related to respiration, speech, swallowing, wheezing, coughing, and sneezing.

52. The electronic device of claim 51, being configured to separate signals associated with the cardiopulmonary system and related processes from those due to body movements.

53. The electronic device of claim 51, being configured to spatiotemporally map movements of the skin at this region of the anatomy onto which the electronic device is attached during cardiac and respiratory activities.

54. The electronic device of claim 51, being configured to continuously measure temperature, heart rate (HR), respiratory rate (RR), activity level, and body orientation, across a range of vigorous activities and conditions.

55. The electronic device of claim 51, being configured to monitor key symptoms of a patient with COVID-19 infection to track progress of recovery and response to therapies in hospital and/or home.

56. The electronic device of claim 51, being configured to measure any of respiratory or motion related digital biomarkers associated with coughing, swallowing, and/or specific motion related activities.

57. The electronic device of claim 56, being configured to assess coughing when the living subject is moving or immobile, and/or to measure muscle motion, when the living subject is moving.

58. The electronic device of claim 39, further comprising a bidirectional wireless communication system electronically coupled to the electronic device and configured to send an output signal from the electronic device to an external device.

59. The electronic device of claim 58, wherein the bidirectional wireless communication system is further configured to deliver commands from the external device to the electronic device.

60. The electronic device of claim 58, wherein the bidirectional wireless communication system comprises a controller that utilizes at least one of near field communication (NFC), Wi-Fi/Internet, Bluetooth, Bluetooth low energy (BLE), and cellular communication protocols for wireless communication.

61. The electronic device of claim 58, further comprising a customized app with a user interface deployed in the external device to allow a user to configure and operate the electronic device for data collection, data transfer, data storage and analysis, wireless charging, and monitoring of user's conditions.

62. The electronic device of claim 61, wherein the customized app is configured to allow time-synchronized operation of the sensor network simultaneously.

63. The electronic device of claim 58, wherein the external device is a mobile device, a computer, or a cloud service.

64. The electronic device of claim 39, further comprising a power module coupled to the sensor network for providing power thereto.

65. The electronic device of claim 64, wherein the power module further comprises a failure prevention element including a short-circuit protection component or a circuit to avoid battery malfunction.

66. The electronic device of claim 64, wherein the power module comprises at least one battery for providing the power.

67. The electronic device of claim 66, wherein the at least one battery is a rechargeable battery.

68. The electronic device of claim 67, wherein the power module further comprises a wireless charging module for wirelessly charging the rechargeable battery.

69. An electronic device for measuring physiological parameters of a living subject, comprising:
a first sensor adapted for detecting a first group of data related to the living subject and a second group of data that is different from the first group of data; and
a second sensor for detecting a third group of data that is substantially similar to the second group of data,
wherein in operation, the first sensor and the second sensor are time-synchronized to allow the third group of data from the second sensor to be used to substantially cancel out the second group of data from the first sensor.

70. The electronic device of claim 69, wherein the first sensor and the second sensor are spatially and mechanically separated from each other.

71. The electronic device of claim 71, wherein the separation of the first sensor and the second sensor is greater than zero and less than a predetermined distance.

72. The electronic device of claim 69, wherein each of the first sensor and the second sensor comprises an inertial measurement unit (IMU), a thermal sensor, a pressure sensor, or optical sensor.

73. The electronic device of claim 69, wherein the first group of data is physiological signals of the living subject, and the second group of data is signals related to ambient, motion and/or vibration at the first sensor.

74. The electronic device of claim 73, wherein the third group of data is signals related to ambient, motion and/or vibration at the second sensor.

75. The electronic device of claim 69, wherein both of the first sensor and second sensor are operably in mechanical communication with the skin of the living subject.

76. The electronic device of claim 75, wherein the first sensor is operably in directly mechanical communication with the skin of the living subject for sensing physiological signals from the body, and the second sensor is operably in indirectly mechanical communication with the skin of the living subject.

77. The electronic device of claim 76, wherein the first sensor and the second sensor are operably in directly mechanical communication with the skin of the living subject for sensing physiological signals from the body to assess pulse transit time.

78. The electronic device of claim 69, being flexible and conformable to the skin with a specific geometrical polarity for mounting in an anatomical location of interest of the living subject.

79. An electronic device for measuring physiological parameters of a living subject, comprising:
a first sensor adapted for detecting a first group of data related to the living subject and a second group of data that is different from the first group of data; and
a second sensor for detecting a third group of data that is substantially similar to the second group of data,
wherein in operation, the first sensor is positioned such that there is a first distance d1 between a center of the first sensor and an area of the living subject where physiological signals of the living subject are measurable;
the second sensor is positioned such that there is a second distance d2 between a center of the second sensor and the center of the first sensor, wherein the second distance d2 is greater than zero and less than a predetermined distance.

80. The electronic device of claim 79, wherein the second sensor is positioned over the first sensor.

81. The electronic device of claim 79, wherein the second sensor is positioned away from the first sensor.

82. The electronic device of claim 79, wherein each of the first sensor and the second sensor comprises an inertial measurement unit (IMU), a thermal sensor, a pressure sensor, or optical sensor.

83. The electronic device of claim 79, wherein the first group of data is physiological signals of the living subject, and the second group of data is signals related to ambient, motion and/or vibration at the first sensor.

84. The electronic device of claim 83, wherein the third group of data is signals related to ambient, motion and/or vibration at the second sensor.

85. The electronic device of claim 79, wherein both of the first sensor and second sensor are operably in mechanical communication with the skin of the living subject.

86. The electronic device of claim 85, wherein the first sensor is operably in directly mechanical communication with the skin of the living subject for sensing physiological signals from the body, and the second sensor is operably in indirectly mechanical communication with the skin of the living subject.

87. The electronic device of claim 85, wherein the first sensor and the second sensor are operably in directly mechanical communication with the skin of the living subject for sensing physiological signals from the body to assess pulse transit time.

88. The electronic device of claim 79, being flexible and conformable to the skin with a specific geometrical polarity for mounting in an anatomical location of interest of the living subject.
